(19) United States
(12) Patent Application Publication
(10) Pub. No.: US 2025/0367381 A1
(43) Pub. Date: Dec. 4, 2025

(54) MEDICAL INJECTIONS AND RELATED DEVICES AND METHODS

(71) Applicant: Gilead Sciences, Inc., Foster City, CA (US)

(72) Inventors: Berk Besen, Cambridge (GB); Stuart Curtis, Cambridge (GB); Patricia Das, Hertfordshire (GB); Walter Goodwin, San Carlos, CA (US); Finlay Knops-McKim, Cambridge (GB); James Paul Oberhauser, Saratoga, CA (US); James Plimmer, Cambridge (GB); Paul Smitheman, Cambridge (GB); Alasdair Young, Foster City, CA (US)

(21) Appl. No.: 19/206,139

(22) Filed: May 13, 2025

Related U.S. Application Data
(60) Provisional application No. 63/647,354, filed on May 14, 2024.

(51) Int. Cl. A61M 5/20; A61M 5/315

(52) U.S. Cl. CPC A61M 5/2053; A61M 5/31511; A61M 5/31568; A61M 5/31578; A61M 2205/3317; A61M 2205/3327; A61M 2205/3368; A61M 2205/3592; A61M 2205/502; A61M 2205/581; A61M 2205/8206

(57) ABSTRACT

Systems, devices, and methods include an autoinjector including a housing; a needle arranged at a distal end of the housing; a container disposed within the housing and configured to contain medicament; and a plunger slidably disposed within the container. A plunger rod is configured to push the plunger through the container to dispense the medicament through the needle when the container contains medicament. The plunger rod includes a magnetic region. A mechanism is configured so that a dispensing movement of the plunger rod causes the mechanism to generate a first magnetic signal. A first sensor is configured to detect the first magnetic signal generated by the mechanism.

### CROSS REFERENCE TO RELATED APPLICATIONS

This application claims priority to U.S. Provisional Application No. 63/647,354, filed May 14, 2024, the entire content of which application is hereby incorporated by reference in its entirety.

### TECHNICAL FIELD

This disclosure relates to medical injections and related devices and methods.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems, devices, and methods can sense a dispensing movement of a plunger rod within an autoinjector. For example, a magnetic region on the plunger rod of the autoinjector can produce signals that can be sensed by a sensing assembly to track the dispensing movement of the plunger rod. The signals can include one or more magnetic signals that indicate that the magnetic region has reached a certain location within the autoinjector. The devices can include a sensing assembly that can determine that the plunger rod has completed the dispensing movement after receiving a predetermined number of signals. In some embodiments, the sensing assembly can be provided as an attachable module configured to reversibly attached to the housing of the autoinjector.

The sensing assembly can also track the dispensing movement of the plunger rod. Tracking the dispensing movement of the plunger rod can be advantageous because the dispensing movement of the plunger rod corresponds to the amount of medicament delivered to the subject (e.g., a patient). If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth with a sufficient insertion force and/or whether the insertion depth is maintained during delivery of the medicament. For example, a needle guard can be configured to retract when an insertion force is applied to the needle and configured to extend when the insertion force is removed. In some embodiments, a mechanical contact between a needle guard and a switch can determine whether the needle has been inserted to a sufficient depth and/or with a sufficient insertion force. Determining that the needle is sufficiently inserted into the patient can be advantageous because if the needle is not sufficiently inserted into the patient, the patient may not receive the medicament properly.

The present disclosure also relates to a composition comprising lenacapavir or a pharmaceutically accepted salt thereof for use in the prevention or treatment of HIV, where the lenacapavir is administered by an autoinjector according to this disclosure. The present disclosure also relates to a use of lenacapavir or a pharmaceutically accepted salt thereof for the manufacture of a medicament for the prevention or treatment of HIV, where the lenacapavir is administered by an autoinjector according to the disclosure.

In a first aspect of the invention, the present disclosure encompasses an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes a magnetic region, and a dispensing movement of the plunger rod generates a first magnetic signal; and a first sensor configured to detect the first magnetic signal as the plunger rod moves.

According to the first aspect of the invention, the first magnetic signal may be indicative of dose progression.

According to the first aspect of the invention, the container may contains the medicament. Additionally or alternatively, the medicament may comprise lenacapavir or a pharmaceutically accepted salt thereof.

According to the first aspect of the invention, the autoinjector may further comprise a gas canister assembly configured to release pressurized gas which, when released, provides a force acting on the plunger rod to push the plunger through the container.

According to the first aspect of the invention, the magnetic region is disposed on a distal end of the plunger rod. Additionally or alternatively, the magnetic region may include a magnet. The plunger rod may be configured to generate a plurality of magnetic signals during the dispensing movement of the plunger rod. Optionally, the first sensor may include an array of sensor units. Each of the sensor units may include, independently, a Hall effect sensor. Additionally or alternatively, the array may include a first sensor unit disposed in proximity to a proximal end of the container and a last sensor unit disposed in proximity to a distal end of the container. Additionally or alternatively, the first magnetic signal may be configured to indicate completion of the dispensing movement of the plunger rod. Optionally, the first sensor unit may be configured to indicate dose start of the medicament, and/or the last sensor unit may be configured to indicate dose end of the medicament. The magnetic region may be configured to generate a corresponding magnetic signal during the dispensing movement of the plunger rod.

The first aspect of the invention, additionally or alternatively, may include an audible clicker configured to produce one or more audible clicks during the dispensing movement of the plunger rod. Optionally, the audible clicker may include a ring surrounding the plunger rod, and the ring includes a deflectable protrusion configured to contact the plunger rod and produce the one or more audible clicks. Further optionally, the plunger rod may include a ridged surface configured to contact the deflectable protrusion of the audible clicker. Each ridge of the ridged surface may be configured to cause the deflectable protrusion to deflect during the dispensing movement of the plunger rod.

According to the first aspect of the invention, the first sensor may be disposed in an attachable module configured to be reversibly attached to the housing. Optionally, the attachable module may include an assembly switch configured to detect attachment of the attachable module to the housing. Further optionally, the assembly switch may be configured to detect attachment of the attachable module to the housing and is further configured to provide power to one or more electronic components of the autoinjector.

The first aspect of the invention, additionally or alternatively, may include an integrated sensor disposed between a proximal end and a distal end of the housing, wherein the integrated sensor comprises a second sensor configured to detect a first electrical signal generated by applying an insertion force to insert the needle into a user and a third sensor is configured to detect an ambient temperature in proximity to the container. Optionally, the first aspect of the invention may include a needle guard contact configured to generate the first electrical signal upon applying the insertion force.

Alternatively, the first aspect of the invention may include a second sensor disposed between a proximal end and a distal end of the housing, wherein the second sensor is configured to detect a first electrical signal generated by applying an insertion force to insert the needle into a user. Optionally, the first aspect of the invention may include a needle guard contact configured to generate the first electrical signal upon applying the insertion force. Additionally or alternatively, the second sensor may include a force sensor. Optionally, the first aspect of the invention may include a third sensor disposed between a proximal end and a distal end of the housing, where the third sensor is configured to detect an ambient temperature in proximity to the container. The third sensor may include a thermistor.

In a second aspect of the invention, the present disclosure encompasses an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes a magnetic region; a mechanism configured so that a dispensing movement of the plunger rod causes the mechanism to generate a first magnetic signal; and a first sensor configured to detect the first magnetic signal generated by the mechanism.

In a third aspect of the invention, the present disclosure encompasses an autoinjector including a housing including a recessed portion; a needle arranged at a distal end of the housing; a needle guard configured to expose the needle when an insertion force is applied to insert the needle into a user; a container disposed within the housing and configured to contain medicament; a plunger slidably disposed within the container; a plunger rod configured to push the plunger through the container to dispense the medicament through the needle when the container contains medicament and when the needle is exposed, where the plunger rod includes a magnetic region, and where a dispensing movement of the plunger rod generates a first magnetic signal; and an attachable module configured to be reversibly attached to the recessed portion of the housing, where the attachable module includes a first sensor configured to detect the first magnetic signal as the plunger rod moves.

In a fourth aspect of the invention, the present disclosure encompasses a system including an autoinjector (e.g., any described herein, such as those of the first, second or third aspects of the invention); and a processor configured to process one or more magnetic signals to track the dispensing movement of the plunger rod.

In a fifth aspect of the invention, the present disclosure encompasses a method of detecting a dispensing movement of a plunger rod within an autoinjector including detecting one or more magnetic signals of a magnetic region as the plunger rod moves during the dispensing movement, where the plunger rod includes the magnetic region disposed on or within the plunger rod, and where the dispensing movement of the plunger rod generates a first magnetic signal.

In a sixth aspect of the invention, the present disclosure encompasses a method of detecting a dispensing movement of a plunger rod within an autoinjector including detecting a magnetic signal generated due to movement of the plunger rod during the dispensing movement.

According to the fifth or sixth aspect of the invention, the plunger rod may be configured to generate a plurality of magnetic signals during the dispensing movement of the plunger rod.

According to the fifth or sixth aspect of the invention, the autoinjector may include an audible clicker configured to produce one or more audible clicks during the dispensing movement of the plunger rod. The audible clicker may include a ring surrounding the plunger rod, and the ring includes a deflectable protrusion configured to contact the plunger rod and produce the one or more audible clicks. Optionally, the plunger rod may include a ridged surface configured to contact the deflectable protrusion of the audible clicker. Further optionally, each ridge of the ridged surface may be configured to cause the deflectable protrusion to deflect during the dispensing movement of the plunger rod.

According to the fifth or sixth aspect of the invention, a first sensor may be disposed between a proximal end and a distal end of the housing. The first sensor may include a magnetic sensor or an array of magnetic sensors. Additionally or alternatively, the first sensor may be disposed on a surface of a printed circuit board.

According to the fifth or sixth aspect of the invention, an integrated sensor may be disposed between a proximal end and a distal end of the housing, and the integrated sensor includes a second sensor configured to detect a first electrical signal generated by applying an insertion force to insert the needle into a user and a third sensor configured to detect an ambient temperature in proximity to the container.

According to the fifth or sixth aspect of the invention, alternatively, a second sensor may be disposed between a proximal end and a distal end of the housing, and wherein the second sensor is configured to detect a first electrical signal generated by applying an insertion force to insert the needle into a user. According to the fifth or sixth aspect of the invention, additionally, a third sensor may be disposed between a proximal end and a distal end of the housing, and the third sensor is configured to detect an ambient temperature in proximity to the container.

The fifth or sixth aspects of the invention may include sending, via a wireless transfer protocol module, signals from a first sensor, if present, an integrated sensor, if present, and/or a second sensor, if present, to a mobile device. Additionally, the fifth or sixth aspects of the invention may include displaying information about the dispensing movement on the mobile device.

The fifth or sixth aspects of the invention may include measuring a temperature of medicament within the autoinjector or an ambient temperature within the autoinjector using a temperature sensor. The fifth or sixth aspects of the invention may further include sending, via a wireless transfer protocol module, signals from the temperature sensor to a mobile device. Additionally or alternatively, the fifth or sixth aspects of the invention may include displaying the temperature of the medicament or the ambient temperature on the mobile device; or indicating that the temperature of the medicament or the ambient temperature is above a threshold temperature for use of the autoinjector; or both.

According to a seventh aspect of the invention, also provided is a composition comprising lenacapavir or a pharmaceutically accepted salt thereof for use in the prevention or treatment of HIV, where the composition is administered via an autoinjector (e.g., any described herein, such as those of the first, second or third aspects of the invention). The administration may be subcutaneous or intramuscular.

According to an eighth aspect of the invention, also provided is the use of lenacapavir or a pharmaceutically accepted salt thereof for the manufacture of a medicament for the prevention or treatment of HIV, wherein the prevention or treatment comprises administering the medicament via an autoinjector (e.g., any described herein, such as those of the first, second or third aspects of the invention). The administration may be subcutaneous or intramuscular.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1A illustrates an example of an autoinjector.

FIG. 1B is a front view of the autoinjector of FIG. 1A.

FIG. 2A is a cut-away view of an example autoinjector.

FIG. 2B is a cross section view of the autoinjector of FIG. 2A.

FIG. 2C is an exploded view of the autoinjector of FIG. 2A.

FIG. 3 is a cross section view of another example autoinjector.

FIG. 4A illustrates an example attachable module of the autoinjector of FIG. 3.

FIG. 4B illustrates an example recess of the autoinjector of FIG. 3.

FIG. 4C illustrates an example plunger rod of the autoinjector of FIG. 3.

FIG. 4D illustrates an example integrated probe of the autoinjector of FIG. 3.

FIG. 5A illustrates side and top views of an example attachable module.

FIG. 5B illustrates an exploded view of the attachable module of FIG. 5A.

FIG. 5C illustrates a bottom view of the attachable module of FIG. 5A.

FIG. 5D illustrates a side view of the attachable module of FIG. 5A for use with a recess of the housing.

FIGS. 6A-C illustrates an example autoinjector during operation of the autoinjector.

FIG. 7 illustrates a process of dispensing a dose of medicament using an example autoinjector.

FIG. 8 is a flow chart for an example method of dispensing a dose using an autoinjector.

FIG. 9 is an example plot of temperature readings of a medicament using an example autoinjector.

FIG. 10 illustrates detected magnetic signals indicating dose progression for an example autoinjector.

Like reference numbers and designations in the various drawings indicate like elements.

### DETAILED DESCRIPTION

The present systems, devices, and methods can sense a dispensing movement of a plunger rod within an autoinjector. For example, a magnetic region on the plunger rod of the autoinjector can produce signals that can be sensed by a sensing assembly to track the dispensing movement of the plunger rod. The signals can include one or more magnetic signals (e.g., which in turn can be converted into electrical measurements) that indicate that a magnetic region has reached a certain location within the autoinjector. The devices can include a sensing assembly that can determine that the plunger rod has completed the dispensing movement. The sensing assembly can also track the dispensing movement of the plunger rod. Tracking the dispensing movement of the plunger rod can be advantageous because the dispensing movement of the plunger rod corresponds to the amount of medicament delivered to the patient. If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth with a sufficient insertion force and/or whether the insertion depth is maintained during delivery of the medicament. For instance, the needle guard can be configured to retract when an insertion force is applied to the needle, thereby undergoing a retraction movement, as well as configured to extend when the insertion force is removed, thereby undergoing an extension movement. By tracking the occurrence of such retraction and extension movements, dose start and dose end events can be detected (e.g., as described herein). Such retraction and extension movements can generate an electrical signal (e.g., by mechanical activation of a switch), which in turn can be sensed by a sensor. Determining that the needle is sufficiently inserted into the patient can be advantageous because if the needle is not sufficiently inserted into the patient, the patient may not receive the medicament properly.

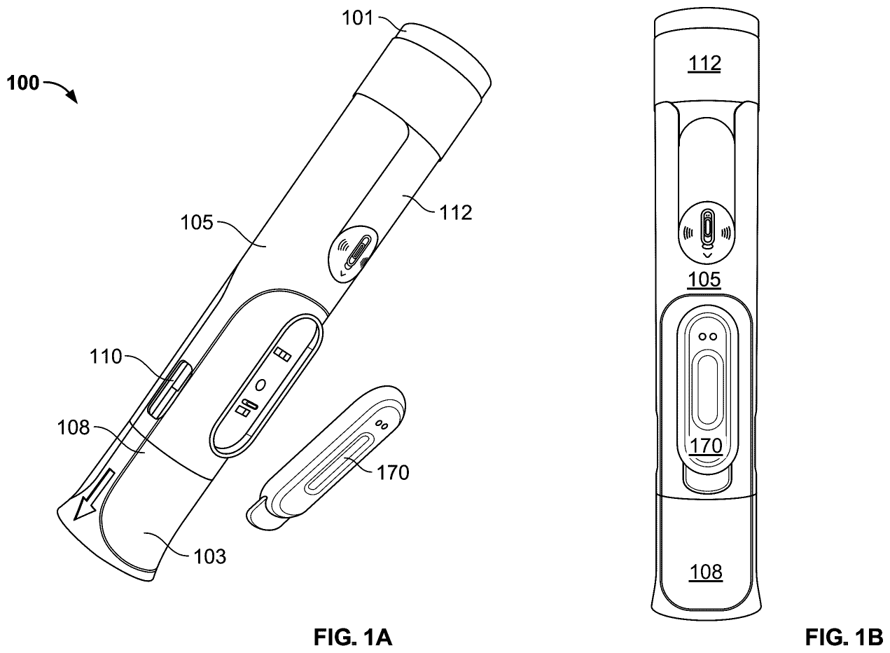

FIG. 1A-B illustrates an autoinjector 100 that can sense a dispensing movement of a plunger rod within the autoinjector 100 and sense whether a needle of the autoinjector 100 is inserted into a patient to a sufficient depth. Alternatively, or in addition, the autoinjector 100 can sense whether an insertion depth of a needle is maintained during dispensing or delivery. The autoinjector 100 includes a housing 105, a proximal end 101 of the housing 105, and a front cap 108 attached to a distal end 103 of the housing 105. The front cap 108 covers a needle assembly configured to be inserted into the user during injection. The user removes the front cap 108 prior to use of the autoinjector 100. The housing 105 can include flanges configured to accommodate the user's fingers.

A sensing assembly can be located somewhere between the proximal end 101 and the distal end 103 of the housing 105. The sensing assembly can sense a dispensing movement of a plunger rod within the autoinjector 100, e.g., to determine that the plunger rod has completed the dispensing movement, as discussed below. The sensing assembly can be provided as an attachable module 170 configured to be reversibly attached to the housing 105.

The housing 105 can include an optional label 112 that provides information about the autoinjector 100. For example, the label 112 can include medicament information, such as the type of medicament, the size of the dose, and the delivery time of the dose. Optionally, the housing 105 does not include a label 112.

The housing 105 also includes an optional window 110, through which a user can see medicament contained within the autoinjector 100, e.g., within a container of the autoinjector 100 (see discussion below). The window 110 may help a user determine whether the autoinjector 100 has been used. Before use of the autoinjector 100, the user can see through the window 110 to determine whether there is medicament within the autoinjector 100, for example, to determine that the autoinjector has not been used. During use of the autoinjector 100, the user may look through the window 110 to determine whether the volume of medicament in the autoinjector 100 is decreasing. After use of the autoinjector 100, the user may look through the window 110 to determine that there is no medicament in the autoinjector 100, for example, to determine that the autoinjector 100 has been used.

Different injection sites, patient age and patient body mass may affect the recommended needle length, and higher viscosity drugs will require a larger diameter needle to prevent the injection force becoming too high for the device. Advantageously, selecting needle gauge based on viscosity of drug being administered can ensure that the full dose of drug is administered without undue strain. Typically, injection force is less than 40 Newtons through needle gauge selection. Preferably, injection force is less than 20 Newtons through needle gauge selection.

Needle gauges disclosed herein are provided in Birmingham Wire Gauge (also known as: Birmingham Gauge or Stubs Iron Wire Gauge), abbreviated as “gauge” or G. In accordance with ISO standard ISO 9626:2016, needle wall thickness designations include Regular Wall, Thin Wall, Extra Thin Wall, and Ultra Thin Wall. Regular Wall thickness is abbreviated to RW. Thin Wall thickness is abbreviated to TW. Extra Thin Wall thickness is abbreviated to ETW. Ultra Thin Wall is abbreviated to UTW. Alternatively, needle wall thickness may be Special Thin Wall; Special Thin Wall thickness is abbreviated as STW. Viscosity is provided in centipoise (cP), where one centipoise is equivalent to one millipascal-second.

The autoinjector 100 may be used for subcutaneous injections, which are directed into fat tissue between the skin and the muscle of the patient. Subcutaneous injections typically involve shorter and narrower needles than intramuscular injections, which are directed into the muscle of a patient. Needles for subcutaneous injections are typically 34-27 gauge and 4-12 mm in insertion depth (needle extension) for subcutaneous injections into the abdomen. Insertion depth for a subcutaneous injection may be 4-8 mm. For subcutaneous injection, needle length may be 8-13 mm. For delivery of 2.25-3 mL dose of liquid (medicament) with a viscosity of up to 5 cP using a needle with a needle length 8-13 mm, needle gauge may be 29 G RW or TW, or 27 G RW. For delivery of 2.25-3 mL dose of liquid (medicament) with a viscosity of up to 30 cP using a needle with a needle length 8-13 mm, needle gauge may be 27 G TW or 25 G RW. For delivery of 2.25-3 mL dose of liquid (medicament) with a viscosity of up to 50 cP using a needle with a needle length 8-13 mm, needle gauge may be 25 G TW. For delivery of 2.25-3 mL dose of liquid (medicament) with a viscosity of up to 160 cP using a needle with a needle length 8-13 mm, needle gauge may be 25 G STW or 23 G RW. For delivery of 2.25-3 mL dose of liquid (medicament) with a viscosity of up to 300 cP using a needle with a needle length 8-13 mm, needle gauge may be 22 G ETW. For delivery of 2.25-3 mL dose of liquid (medicament) with a viscosity of up to 600 cP using a needle with a needle length 8-13 mm, needle gauge may be 18 G ETW or 18 G UTW. For delivery of 1 mL dose of liquid (medicament) with a viscosity of up to 10 cP using a needle with a needle length 8-13 mm, needle gauge may be 29 G RW or TW, or 27 G RW. For delivery of 1 mL dose of liquid (medicament) with a viscosity of up to 30 cP using a needle with a needle length 8-13 mm, needle gauge may be 27 G TW or 25 G RW. For delivery of 1 mL dose of liquid (medicament) with a viscosity of up to 80 cP using a needle with a needle length 8-13 mm, needle gauge may be 25 G TW. For delivery of 1 mL dose of liquid (medicament) with a viscosity of up to 350 cP using a needle with a needle length 8-13 mm, needle gauge may be 25 G STW or 23 G RW. For delivery of 1 mL dose of liquid (medicament) with a viscosity of up to 600 cP using a needle with a needle length 8-13 mm, needle gauge may be 18-22 G ETW.

The autoinjector 100 may be used for intramuscular injections. Needles for intramuscular injections on adults are typically 25-20 gauge and 15-25 mm in insertion depth (needle extension). Alternatively, insertion depth for an intramuscular injection may be 25-50 mm. For intramuscular injection, needle length may be 1-1.5 inches (25.4-38.1 mm). For delivery of 2.25-3 mL dose of liquid (medicament) with a viscosity of up to 1 cP using a needle with a needle length 25.4-38.1 mm, needle gauge may be 29 G RW or TW, or 27 G RW. For delivery of 2.25-3 mL dose of liquid (medicament) with a viscosity of up to 5 cP using a needle with a needle length 25.4-38.1 mm, needle gauge may be 27 G TW or 25 G RW. For delivery of 2.25-3 mL dose of liquid (medicament) with a viscosity of up to 10 cP using a needle with a needle length 25.4-38.1 mm, needle gauge may be 25 G TW. For delivery of 2.25-3 mL dose of liquid (medicament) with a viscosity of up to 40 cP using a needle with a needle length 25.4-38.1 mm, needle gauge may be 25 G STW or 23 G RW. For delivery of 2.25-3 mL dose of liquid (medicament) with a viscosity of up to 200 cP using a needle with a needle length 25.4-38.1 mm, needle gauge may be 22 G ETW. For delivery of 2.25-3 mL dose of liquid (medicament) with a viscosity of up to 600 cP using a needle with a needle length 25.4-38.1 mm, needle gauge may be 18 G ETW. For delivery of 1 mL dose of liquid (medicament) with a viscosity of up to 5 cP using a needle with a needle length 25.4-38.1 mm, needle gauge may be 29 G RW or TW, or 27 G RW. For delivery of 1 mL dose of liquid (medicament) with a viscosity of up to 10 cP using a needle with a needle length 25.4-38.1 mm, needle gauge may be 27 G TW or 25 G RW. For delivery of 1 mL dose of liquid (medicament) with a viscosity of up to 30 cP using a needle with a needle length 25.4-38.1 mm, needle gauge may be 25 G TW. For delivery of 1 mL dose of liquid (medicament) with a viscosity of up to 50 cP using a needle with a needle length 25.4-38.1 mm, needle gauge may be 25 G STW or 23 G RW. For delivery of 1 mL dose of liquid (medicament) with a viscosity of greater than 50 cP using a needle with a needle length 25.4-38.1 mm, needle gauge may be 18-22 G ETW.

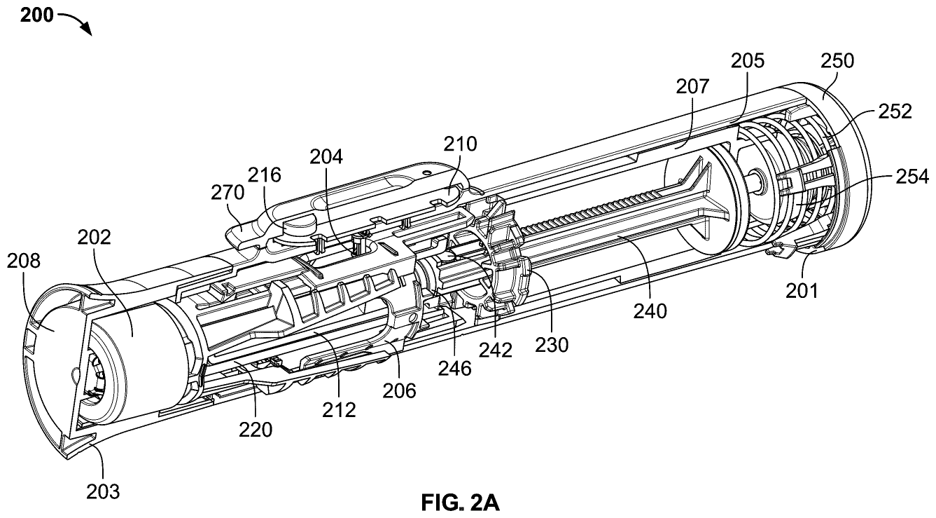

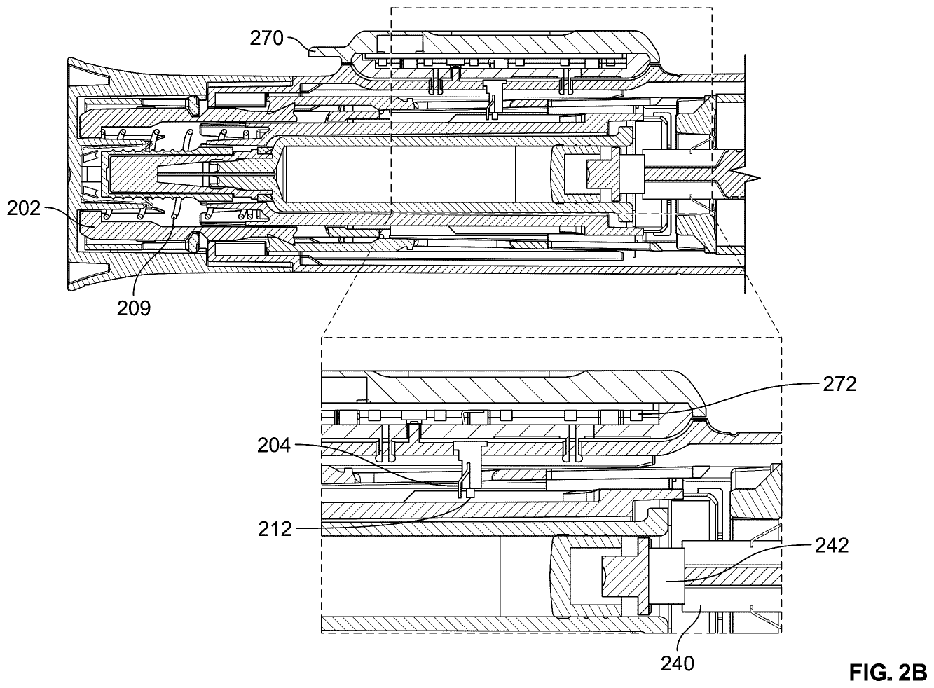

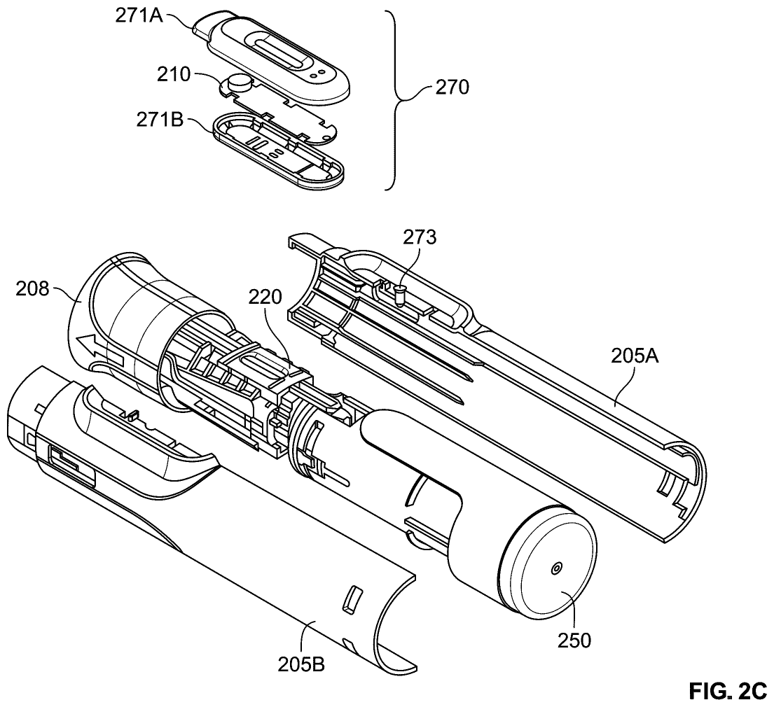

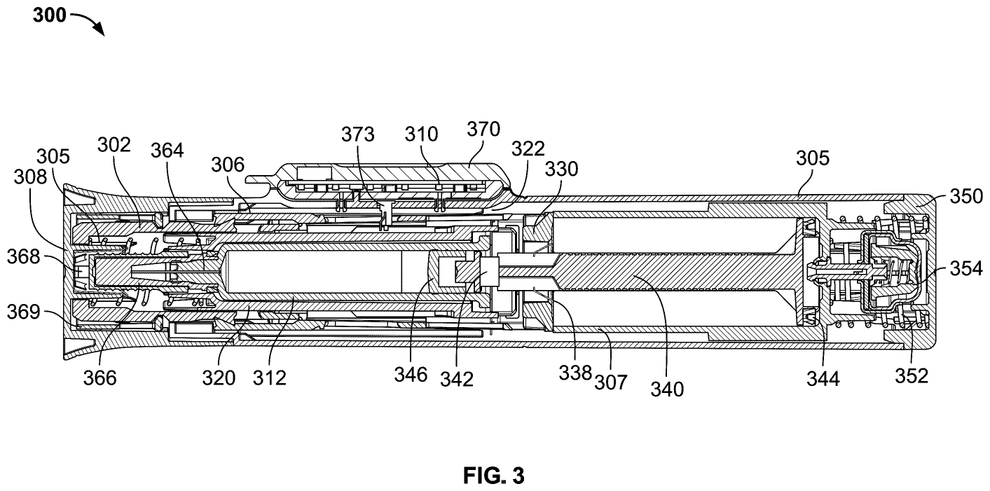

FIG. 2A is a cut-away view of an example autoinjector 200. The autoinjector 200 includes a housing 205 and a front cap 208. The front cap 208 is disposed at a distal end 203 of the housing 205. When installed on the autoinjector 200 (as shown in FIG. 2A), the front cap 208 covers the needle guard 202. The user removes the front cap 208 from the autoinjector 200 prior to use of the autoinjector 200. The needle guard 202 protects the needle when the autoinjector 200 is not in use. The autoinjector 200 includes a container 212 (e.g., a syringe) within the housing 205 for holding a medicament to be injected. The container 212 may have an internal volume of 1.5-3 mL. The container 212 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 212 is disposed in a syringe carrier 220. Optionally, a syringe backstop can hold and maintain the position of the container 212 within the housing 205. A plunger 246 is slidably disposed within the container 212. A plunger rod 240 is configured to push the plunger 246 through the container 212 to dispense the medicament through the needle. The plunger rod 240 protrudes through a lock ring 230. The plunger rod 240 and lock ring 230 are disposed within a delivery chamber 207.

At a proximal end 201 of the autoinjector 200, a rear case 250 is coupled to the housing 205. The proximal end 201 also includes a spring 252 (e.g., an anti-rattle spring) and a gas canister assembly 254. The spring 252 is disposed between the rear case 250 and the delivery chamber 207. The spring 252 biases the delivery chamber 207 toward the distal end 203. The gas canister assembly 254 includes pressurized gas that when released provides a force acting on the plunger rod 240 to push the plunger 246 through the container 212 in a dispensing movement to dispense the medicament. During a dispensing movement, the pressurized gas is contained in the delivery chamber 207, and an optional piston seal can reduce leakage around the plunger rod 240.

Activation of the gas canister assembly 254 can occur by a user fully depressing the needle guard 202 into the housing 205 (e.g., by pressing the needle guard 202 and the autoinjector 200 against his or her skin), such that the needle guard 202 retracts (e.g., undergoing a retraction movement) and moves the transfer sleeve 206 proximally. In turn, this causes all of the internal components (e.g., including the delivery chamber 207) to move a relatively small proximal distance against the bias of the spring 252 disposed at a proximal end 201 of the autoinjector 200. For example, the relatively small proximal distance can be a distance of 1-10 millimeters. Upon moving such a relatively small proximal distance, the gas canister assembly 254 can be activated (e.g., by movement of a firing pin that pierces an end of a gas canister within the gas canister assembly 254), thereby releasing compressed gas from within. The compressed gas can include, for example, argon, carbon dioxide, krypton, xenon, etc. Optionally, depression of the needle guard 202 can cause an internal component to contact a sensing assembly including a switch, a transducer, and/or a force sensor, such that a switch can sense a retraction movement of the needle guard, a transducer can convert a signal input to an electrical signal, and/or a force sensor can measure the insertion force of the needle into the user (e.g., as described herein).

The plunger rod 240 can include a magnetic region (e.g., magnet 242) disposed at a distal end of the plunger. The magnetic region may be disposed in proximity to the plunger 246 and on a surface of the plunger rod 240. A magnetic region can include one or more magnets. The magnet(s) can have any useful regular and irregular shape(s), size(s), material(s), and/or location(s) on the plunger rod. The magnetic region can be located in a region to determine dose progression and when the start and end of the dose gas been reached. The magnetic region can include a magnetic material (e.g., a permanent magnet, a neodymium magnet (e.g., neodymium-iron-boron magnet), a samarium-cobalt magnet, a ferrite magnet, a platinum-cobalt magnet, a chromium-cobalt-iron magnet, a ceramic magnet, and/or an aluminum-nickel-cobalt magnet) and/or a metal (e.g., iron, nickel, cobalt, gadolinium, neodymium, samarium, steel, magnetite, a ferrite, as well other metals, alloys, or composites capable of being magnetized).

A region of the plunger rod can include a holding surface that allows for attachment of a magnet. The holding surface can include an adhesive (e.g., a pressure sensitive adhesive), a metal, a magnetic material, or a combination of these. The magnetic region and/or the holding surface can be disposed on the plunger rod in any useful manner, such as soldering, adhering, sintering, depositing, plating, coating, painting, spraying, printing, etching, patterning, annealing, or combinations thereof.

The sensing assembly of the autoinjector 200 can include a main PCB 210 and a battery 216. The main PCB 210 includes electronics to sense magnetic signals, electrical signals (e.g., a voltage sensor, a resistance sensor, a force sensor), process data, and/or communicate with an external device (e.g., a mobile device, smartphone, or tablet) through a wireless communication protocol (e.g., short range radio communication, near field communication, wireless transfer protocol, Wi-Fi). The battery 216 provides power to the main PCB 210 through a connection (e.g., wiring). The main PCB 210 and the battery PCB 216 can be provided within an attachable module 270 configured to be reversibly attached to the housing 205.

The needle guard 202 can interact with a needle guard contact 204. As seen in FIG. 2A-2B, the needle guard contact 204 can be disposed within the housing 205. The needle guard contact 204 is configured to contact an arm of the needle guard 202 when it is in a retracted position. Contact between the arm of the needle guard contact 202 and the needle guard contact 204 indicate that the needle of the autoinjector 200 is exposed, and the autoinjector 200 is ready for the dosing process to begin. The location and configuration of the needle guard contact 204 can be optimized to balance exposure of the needle, insertion depth of the needle into the patient's skin, and/or dosing initiation.

The contact between the arm of the needle guard 202 and the needle guard contact 204 can be detected by a sensor (e.g., a voltage sensor, resistance sensor, or other sensor configured to detect an electrical signal as the needle guard moves). The needle guard contact 204 may be a mechanical switch, in which the arm of the needle guard 202 triggers the mechanical switch and provides an electrical signal that can be detected by a sensor (e.g., a voltage sensor, resistance sensor, or other sensor configured to detect an electrical signal). The arm of the needle guard 202 may include a contact, which in turn can interact with the needle guard contact 204 to provide an electrical signal that can be detected by the sensor (e.g., a voltage sensor, resistance sensor, or other sensor configured to detect an electrical signal). One or more sensors can be provided in any useful manner. One or more sensors may be disposed in an attachable module 270. Such sensors can include a voltage sensor, resistance sensor, other sensor configured to detect an electrical signal, a temperature sensor 212, and/or one or more magnetic sensors 272, or combinations of any of these.

As seen in FIG. 2B, a needle guard spring 209 is disposed between the needle guard 202 and the syringe carrier 220. The needle guard spring 209 biases the needle guard 202 toward the distal end 203 of the housing, causing the needle guard 202 to extend from the housing 205 and cover the needle. Retraction of the needle guard 202 compresses the needle guard spring 209. The needle guard 202 can be retracted by exerting a force on the distal end 203. Contact between a portion of the needle guard 202 and the needle guard contact 204 can indicate that a sufficient force has been applied to the autoinjector 200 to retract the needle guard 202 and expose the needle. Upon removing the sufficient force applied to the autoinjector 200 (e.g., after the dose is dispensed), the needle guard 202 can then extend from the housing to cover the needle.

As also seen in FIG. 2B, one or more magnetic sensors can be provided (e.g., in the attachable module) to detect one or more magnetic signals. In turn, a magnetic signal can be provided by a magnet 242 provided at an end of the plunger 240. As the plunger rod 240 undergoes a displacement movement, the attached magnet 242 also travels along with the plunger rod 240. By detecting a presence of the magnet 242 during travel, the dispensing movement can be tracked and monitor. A plurality of magnetic sensors 272 can be provided in proximity to the path of travel during the dispensing movement, in which the location of each magnetic sensor can be distributed throughout this path. The detection of a magnetic signal by the magnet 242 by a magnetic sensor at a certain location can indicate dose progression. A plurality of magnetic sensors may be provided within an array, in which spacing between each magnetic sensor can be regular or irregular and in which any useful arrangement can be employed (e.g. linear, ring, etc.). The magnetic sensor can include a Hall effect sensor and the like.

As the magnet 242 and the plunger rod 240 undergoes the dispensing movement, a plurality of magnetic signals can be generated along the path of travel by way of the magnet 242 interacting with one or more of the plurality of magnetic sensors. A location of the magnetic signal can be determined by detecting which magnetic sensor (e.g., in an array) senses that signal. Furthermore, the magnitude of the magnetic signal can also indicate the location of the signal. For example, a signal's magnitude can depend on the distance between the magnet and the magnetic sensor, as well as the sensitivity of the magnetic sensor. Thus, a plurality of signals (e.g., having same or different signal magnitude) from an array of magnetic sensors can be used to determine a location of the magnet (and dose progression) along the travel path of the plunger rod.

Components for the autoinjector can be provided and assembled in any useful manner to provide an autoinjector. As seen in FIG. 2C, the autoinjector can include a first housing portion 205A and a second housing portion 205B that can be assembled to form a housing 205. An integrated probe 273 can be provided within the housing. The integrated probe may include the needle guard contact 204 and a temperature sensor 212. In turn, the housing 205 can surround an internal assembly including a syringe carrier 220, a front cap 208, and a rear case 250. An upper case 271A and a lower case 271B can be assembled around the main PCB 210, thereby forming an attachable module 270 that can be configured to be reversibly attached to the housing 205.

FIG. 3 is a cross-section view of yet another example autoinjector 300. The autoinjector 300 includes a housing 305 and front cap 308 coupled to a distal end of the housing 305. The front cap 308 includes a needle shield remover 368 that can remove a needle shield 366 from a needle 364 when the front cap 308 is removed from the housing 305. The front cap 308 also includes an anti-drop ring 369 to reduce the likelihood of accidental removal of the front cap 308 due to the autoinjector 300 being dropped by a user.

The needle 364 is connected to container 312. The needle shield 366 and needle guard 302 protect the needle 364 prior to use of the autoinjector 300. The needle guard 302 is biased away from the container 312 by the needle guard spring 305. The needle guard 302 includes a portion (e.g., an arm) that can contact a needle guard contact (e.g., of an integrated probe 373) during use of the autoinjector 300 to indicate that the needle 364 is inserted to an appropriate depth in the patient.

The container 312 can include a medicament to be injected into a patient. The container 312 may have an internal volume of 1.5-3 mL. The container 312 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 312 is held in place within the autoinjector 300 by syringe carrier 320. A syringe backstop 322 holds the syringe carrier 320 in place restricting movement of the syringe carrier 320 toward the rear case 350.

A transfer sleeve 306 is positioned around the syringe carrier 322. The transfer sleeve 306 interfaces between the needle guard 302 and the delivery chamber 307.

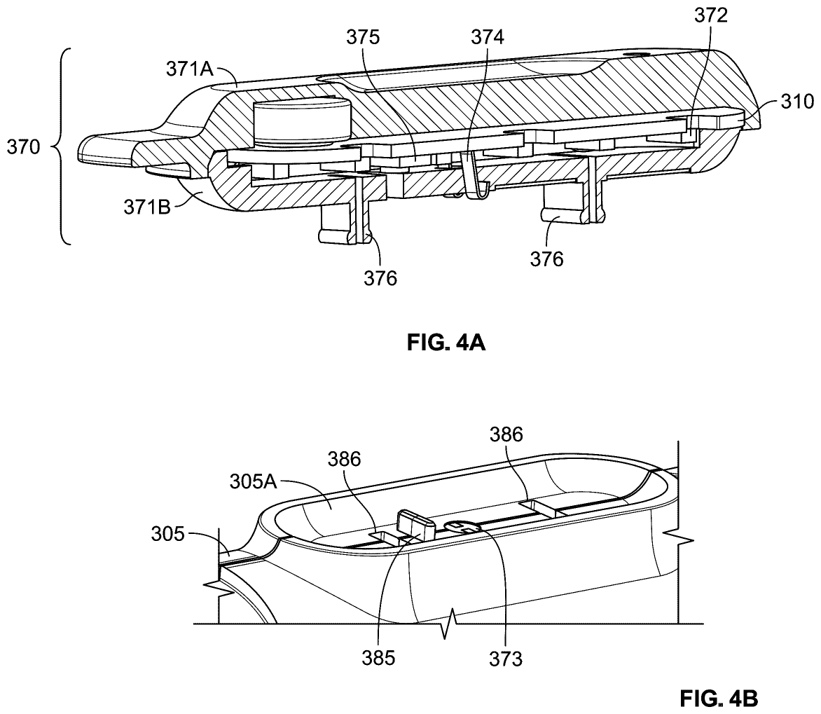

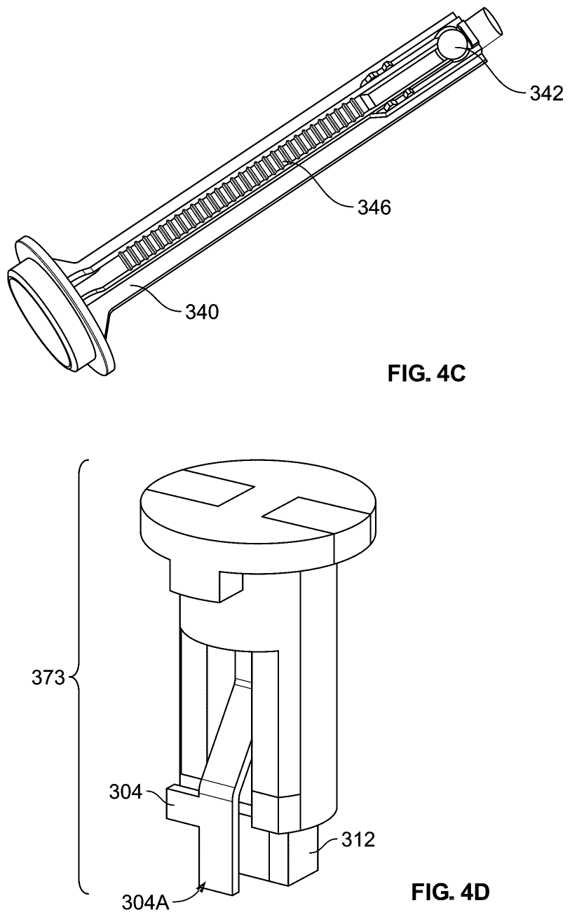

The main PCB 310 is located in an attachable module 370 formed by assembling an upper case 371A and a lower case 371B. The attachable module 370 configured to be reversibly attached to the housing 305. As seen in FIG. 4A-4B, attachment can use one or more clips 376 or other fasteners for reversible attachment between the clips 376 of the attachable module 370 and the slots 386 of the housing. An assembly switch 375 can be configured to detect attachment of the attachable module 370 to a recess 305A of the housing 305. Contact can be made between the assembly switch 375 of the attachable module 370 and the assembly actuator 385 of the housing 305.

As seen in FIG. 4A, sensors (e.g., any herein, such as a magnetic sensor) can be provided in the attachable module 370, such as one or more magnetic sensors 372 disposed on a surface of the main PCB 310. Other examples of sensors include Alternatively, or additionally, the main PCB 310 includes one or more resistance sensors. The main PCB 310 may include a thermistor to measure an ambient temperature in the area in proximity to the container that includes the medicament. The main PCB 310 may include a force sensor to measure an insertion force to insert a needle into a user.

An electrical connection between the attachable module 370 and components within the housing 305 can be formed in any useful manner. A probe contact 374 can be provided in the attachable module to form an electrical connection with a sensor, such as an integrated probe 373. As seen in FIG. 4D, the integrated probe can include a needle guard contact 304 configured to generate an electrical signal upon applying a force 304A and a temperature sensor 312 configured to provide a temperature measurement (e.g., an ambient temperature) in proximity to the container 312 containing a medicament. The needle guard contact may be a switch (e.g., a sprung sheet-metal switch that closes when contacted by a feature of a needle guard). The needle guard contact may be an electrical contract, which forms an electrical connection with a conductive surface disposed on a feature of the needle guard.

A thermistor can be used to measure (indirectly or directly) the temperature (e.g., an approximate temperature) of a medicament included in the container 312 in the syringe carrier. Direct temperature of the medicament may impact sterility of the medicament. Indirect temperature reading of the medicament (e.g., by detecting an ambient temperature within the autoinjector and/or in proximity to the medicament) may be sufficient for dose initiation. A reading from the thermistor can be used to determine that the medicament is at a specified temperature to be injected into a patient. For example, the thermistor can measure the ambient temperature in the region near the syringe carrier and the temperature of the medicament can be inferred based on the ambient temperature near the syringe carrier. Optionally, the main PCB 310 can restrict dispensing of the medicament until the thermistor indicates that an appropriate temperature (e.g., ambient temperature) of the medicament has been reached.

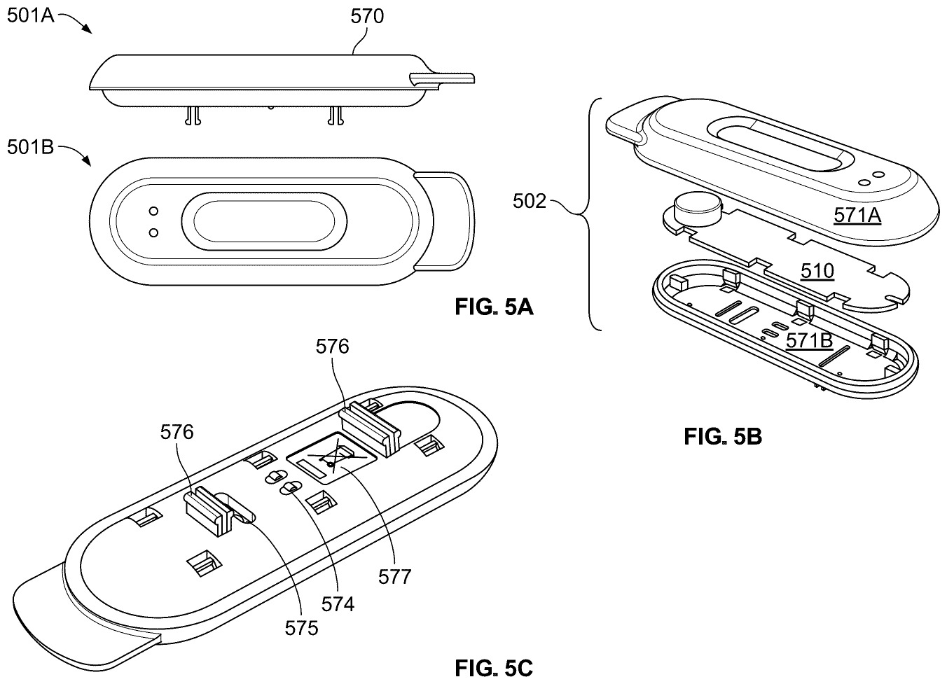

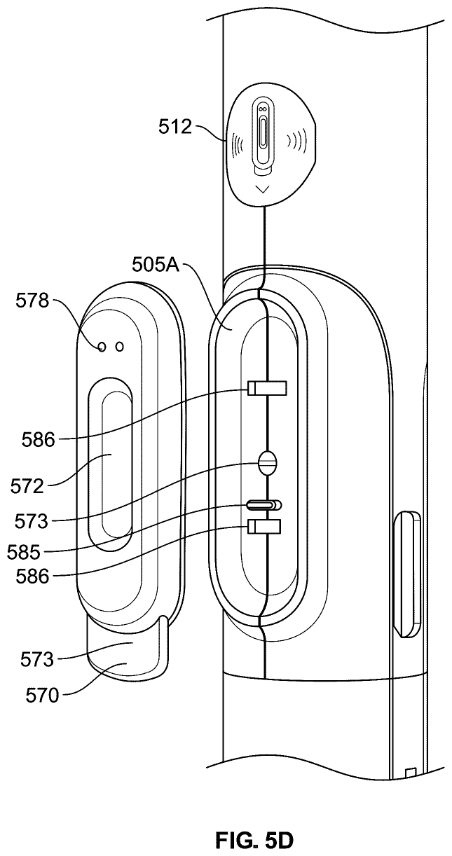

FIGS. 5A-5D show another example attachable module 570, which is formed by assembling an upper case 571A and a lower case 571B around a main 510B to provide a snap fit assembly 502 in a side view 501A and a top view 501B. The attachable module 570 can facilitate the attaching to and detaching from the housing, such as a recess 505A of the housing. Such attachable modules can include a tab portion 573 for the user to more easily removal of the attachable module 570, an indented portion 572 for the user to press the attachable module 570 into the recess 505A, and the like.

For reversible attachment to the housing, the attachable module 570 includes poke-yoke push fit clips 576 to be inserted into the poke-yoke slots 586 of the housing. An assembly switch 575 can provide wake-up access upon contact with the assembly actuator/wake-up pin 585 of the housing, and probe contacts 574 can provide power and/or an electrical connection to the probe 573 within the housing. Optional disposal instructions 577 can be provided to dispose the attachable module 570 having a battery or other power source. Alternatively, the attachable module 570 can be recycled or reused with another autoinjector. Optional instructions or illustrations 512 can be provided on the housing for attaching the attachable module 570, and optional status indicators 578 can provide indications of successful attachment of the attachable module 570.

As seen in FIG. 3, the plunger rod 340 protrudes through a lock ring 330 to exert a force on the plunger 346 that is slidably disposed within the container 312 to dispense the contents of the container 312. As seen in FIG. 4C, the plunger rod 340 provides a magnet 342 in proximity to the plunger 346 disposed at the distal end of the plunger rod 340. The magnet can be positioned at any location and surface of the plunger rod. A plurality of magnets may be employed.

The delivery chamber 307 is disposed between the lock ring 330 and the gas canister assembly 354. The delivery chamber 307 includes the plunger rod 340. The piston seal 344 forms a seal between the plunger rod 340 and sides of the delivery chamber 307 to reduce leakage of the pressurized gas from the gas canister assembly 354 past the plunger rod 340. The gas canister assembly 354 is disposed between the delivery chamber 307 and the rear case 350. A spring 352 is disposed around the gas canister assembly 354. The spring 352 biases the delivery chamber 307 toward the needle 364.

As seen in FIG. 3, the autoinjector 300 can also include a circular clicker 338 (e.g., a ring configured to include an opening therein) disposed on a proximal side of the lock ring 330. The circular clicker 338 interfaces with the plunger rod 340. As can be seen, openings and other structural elements (e.g., contacts, stops, arms, etc.) for the lock ring 230 and the circular clicker 338 can be configured to minimize impeding a pathway for the dispensing movement of the plunger rod 340. As the plunger rod 340 moves through the circular clicker 338 during a dispensing movement, the circular clicker 338 emits one or more audible click sounds that can be used to track the dispensing movement. For example, the circular clicker 338 can have a deflectable protrusion configured to contact a surface of the plunger rod 340. The dispensing movement of the plunger rod 340 can deflect the deflectable protrusion to cause the circular clicker 338 to produce one or more audible clicks, vibrations or the like. A surface of the plunger rod 340 can be configured to deflect the deflectable protrusion of the circular clicker 338.

The circular clicker 338 can include multiple deflectable protrusions. Where the circular clicker 338 includes multiple deflectable protrusions, the plunger rod 340 can include multiple surfaces configured to deflect the deflectable protrusions. For example, and without limitation, the plunger rod 340 can include multiple surfaces configured to deflect the deflectable protrusions, in which each surface can in turn include a plurality of ridges, bumps, projections, or other non-continuous structures along the surface to provide a ridged surface or other non-continuous surface (see discussion below). Alternatively, the circular clicker 338 may include a single deflectable protrusion. Where the circular clicker 338 includes a single deflectable protrusion, the plunger rod 340 can include a single surface configured to deflect the deflectable protrusion. Any number of deflectable protrusions can be employed to produce one or more clicks (e.g., a single deflectable protrusion or multiple deflectable protrusions). In one non-limiting example, a single deflectable protrusion can be employed to reduce the possibility of interference between multiple audible clicks. In another non-limiting example, multiple deflectable protrusions can be employed to operate in sync to provide a more distinct click sound, in which interference can be minimized, e.g., by control of tolerancing and/or alignment.

The circular clicker can be a portion of the autoinjector housing. For example, the autoinjector housing can include a deflectable protrusion that contacts the plunger rod 340. The plunger rod 340 can include one or more deflectable protrusions, and the housing (or another internal component of the autoinjector) includes a surface configured to deflect the one or more deflectable protrusions.

As seen in FIG. 4D, a plunger rod 340 includes a surface having one or more ridges 346, in which a ridged surface can be configured to contact a deflectable protrusion (e.g., of an audible clicker, of a housing, or another appropriate component of the autoinjector). The ridged surface can include any appropriate number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of ridges. Each ridge of a ridged surface can deflect the deflectable protrusion to cause the deflectable protrusion to produce a signal (e.g., an audible click, a vibration, etc.) that can be sensed by a sensing assembly. For example, an example sensing output can include multiple signals, in which each signal corresponds to a ridge of a ridged surface. In turn, the number of signals can be used to track the number of ridges along the plunger rod 340 that passed by the circular clicker during a dispensing movement of the plunger rod 340. For example, a sensing assembly can use the received signals to determine that the plunger rod 340 has completed the dispensing movement after a predetermined number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of signals. The ridged surface can be any non-continuous surface configured to provide a plurality of non-continuous structures (e.g., bumps, projections, ribs, and the like upon that surface).

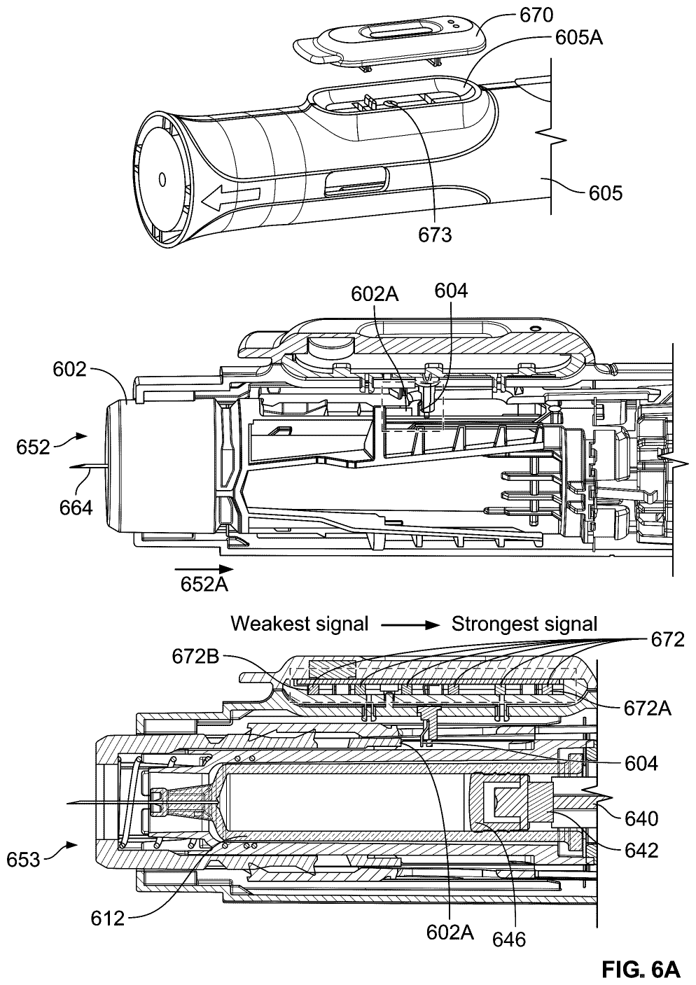

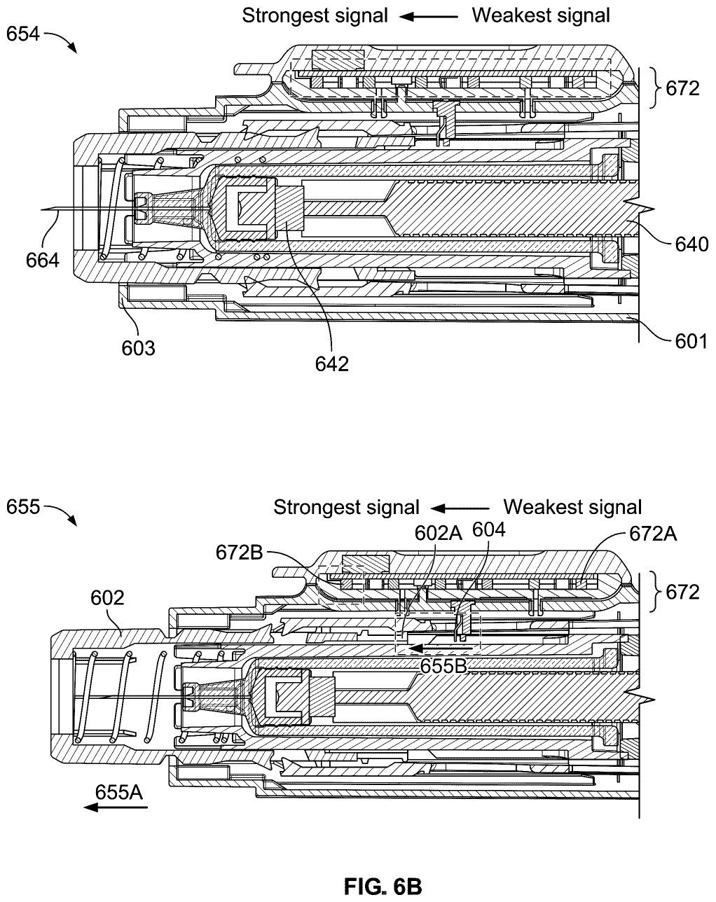

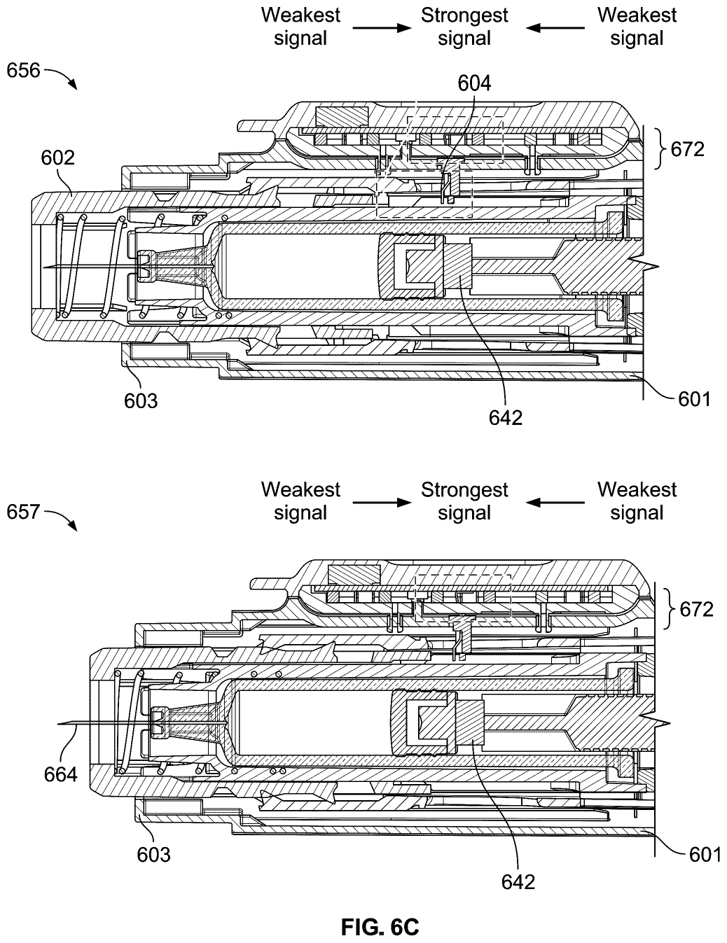

FIG. 6A-6C illustrates a dispensing movement of an example autoinjector. At position 651, an attachable module 670 is fit into a recess 605A of the housing 605. Then, the autoinjector is pressed against the skin of a user.

At position 652, a force 652A is exerted against the distal end of the needle guard 602 to expose the needle 664 through the needle guard 602. The arm 602A of the needle guard 602 moves toward the needle guard contact 604, which can be provided within an integrated probe 673. The contact between the needle guard arm 602A and the needle guard contact 604 indicates that the needle 664 is exposed a specified distance beyond the distal end of the needle guard 602 (e.g., the needle 664 is inserted to an appropriate depth in a patient). The movement of the needle guard 602 activates the gas canister assembly to begin releasing pressurized gas into the delivery chamber. The lock ring and plunger rod 640 translate toward the distal end of the autoinjector. The needle guard arm 602A contacts the needle guard contact 604 indicating that the dispensing movement has started.

At position 653, the plunger rod 640 slides through the lock ring and into the container 612. The magnet 642 on the plunger rod 840 is provided in proximity to the magnetic sensors 672. One or more magnetic sensors 672 can be configured to track certain events during dose progression. For example, and without limitation, depending on the magnitude of the magnetic signal sensed by the magnetic sensors 672, the dose progression can be monitors. For example, the most proximal magnetic sensor 672A in the array of magnetic sensors 672 will initially provide the strongest signal as the magnet 642 is closest to the proximal magnetic sensor 672A at dose start. In contrast, the most distal magnetic sensor 672B in the array of magnetic sensors 672 will initially provide the weakest signal as the magnet 642 is farthest from the distal magnetic sensor 672B at dose start. Magnetic sensors disposed between the proximal magnetic sensor 672A and distal magnetic sensor 672B will provide signals of intermediate strength.

The plunger rod 640 can include ridges that can interface with an audible clicker (e.g., by way of a circular clicker in proximity to the lock ring, as described herein) to generate audible clicks as the dose progresses. The audible clicks form an alternate way (or an additional way with the magnetic signals from the magnet 642) of tracking dose progress during the dispensing movement.

At position 654, the plunger rod 640 slides to the distal end 603. The magnet 642 on the plunger rod 840 is provided in proximity to the distal magnetic sensor 672B, which now provides the strongest signal within the array of magnetic sensors 672. This signal indicates that the dispensing movement has ended and that the end of the dose has been achieved.

The autoinjector can be removed from the skin of the user, such that the applied force is now removed from the needle guard 602. This results in an extension movement 655A of the needle guard 602 to shield the needle 664, and this extension movement in turn moves 655B the needle guard arm 602A away from the needle guard contact 604 to break the switch or electrical contact. A signal generated by breaking contact with the needle guard contact 604 indicates the end of injection. A combination of this signal from the needle guard contact 604 with the magnetic signal indicating end of dose provides an indication of successful delivery of the medicament.

At position 656, premature lift off of the autoinjector results in an extension movement of the needle guard 602 to shield the needle 664, and this extension movement in turn moves the needle guard arm 602A away from the needle guard contact 604 to break the switch or electrical contact. However, the magnetic sensors 672 do not indicate an end of dose, as the strongest signal is sensed between the most proximal magnetic sensor 672A at the proximal end 601 and the most distal magnetic sensor 672B at the distal end 603. Such a signal pattern indicates that the magnet 642 (and the plunger rod 640 to which it is attached) has not traveled the full dispensing movement.

At position 657, a stalled dose results in failure of the plunger rod 640 to travel the full dispensing movement (e.g., through blockage of the needle 664 or within the container 612). Here, the needle guard 602 is still retracted, and the needle 664 is still exposed. Thus, needle guard contact 604 indicates an activated the switch or active electrical contact. However, the magnetic sensors 672 still do not indicate an end of dose, as the strongest signal is sensed between the most proximal magnetic sensor 672A at the proximal end 601 and the most distal magnetic sensor 672B at the distal end 603. Such a signal pattern indicates that the magnet 642 (and the plunger rod 640 to which it is attached) has not traveled the full dispensing movement.

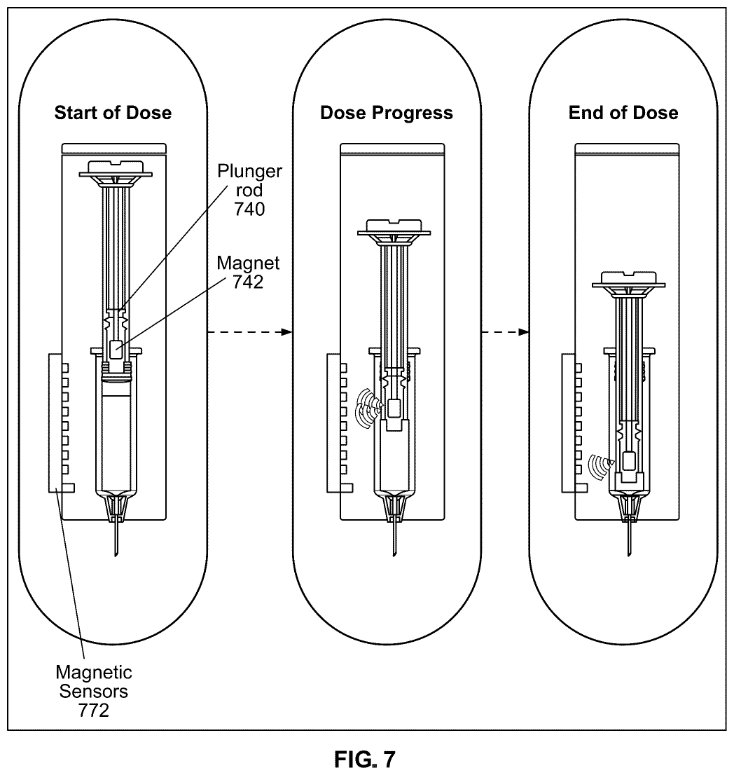

FIG. 7 provides a schematic of dose progression with another example autoinjector. At the start of dose, the magnet 742 disposed at the distal end of the plunger 740 can provide a magnetic signal to at least the most proximal magnetic sensor in the array of magnetic sensors 772. As the dose progresses, the magnet 742 provides a plurality of magnetic signals to be sensed by the magnetic sensors 772. Each magnetic sensor can provide same or different strength signals, depending on the distance between the magnet and the respective magnetic sensor. Such magnetic signals can be used to track the progress of the dispensing movement. The plunger rod 740 may include a plurality of ridges to deflect portions of an audible clicker, which in turn emits an audible click for each ridge that passes the audible clicker disposed in proximity to the lock ring. The audible clicker can form a secondary indication of the dose progress. At the end of the dose, the magnet 742 can provide a magnetic signal to at least the most distal magnetic sensor in the array of magnetic sensors 772.

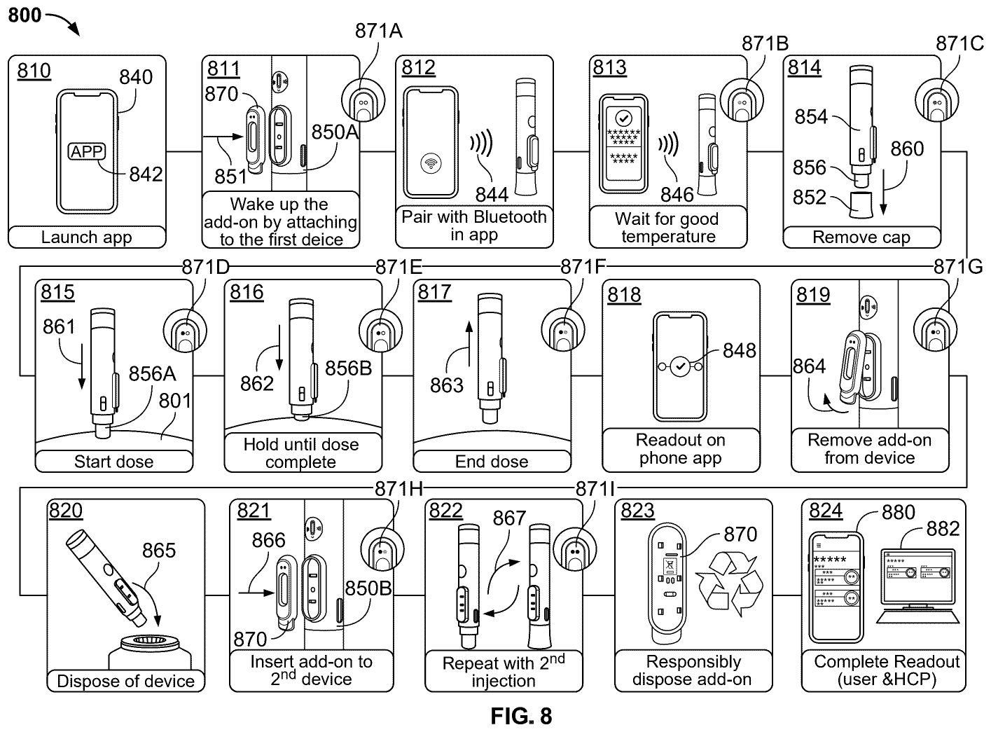

FIG. 8 illustrates an example method 800 of using an autoinjector. The autoinjector can be similar to, e.g., the autoinjectors 100, 200, and 300 described above. In the method 800, a first autoinjector 850A is in communication with a mobile device 840. For example, the autoinjector can be in communication with the mobile device 840 via a wireless transfer protocol module or near field communication protocol (see discussion above). In step 810, the method 800 includes launching an app 842 and/or website on the mobile device 840. In step 811, the method 800 includes waking up electronics within the attachment module 870 and/or the housing by attaching 851 the attachment module 870 to the housing of the autoinjector 850A. Optionally, waking up can include pressing a button on the housing of the autoinjector 850A, the app 842, and/or website on the mobile device 840. The button may not be activated, but, optionally, activation of an assembly switch, force sensor, or electrical contact between elements in the autoinjector can wake up the electronics in the autoinjector. The electronics in the autoinjector (e.g., in the attachment module 870 and/or the housing) can include, e.g., a PCB, a magnetic sensor, a voltage sensor, a resistance sensor, a temperature sensor (e.g., a thermistor), a force sensor (e.g., a force sensitive resistor, a load cell, a strain gauge, a force sense capacitor), a vibration sensor (e.g., an accelerometer, a microphone (e.g., a contact microphone or an air microphone), a displacement sensor, a velocity sensor)), a switch (e.g., a needle guard switch and/or an assembly switch), and the like. Optionally, the magnetic sensor can sense a movement of a needle guard and/or a dispensing movement of a plunger rod that provides a magnetic signal, as discussed above; the temperature sensor can sense a temperature in proximity to the medicament; the force sensor can sense an insertion force used to insert the needle of the autoinjector a sufficient distance into the patient; and/or the vibration sensor can sense a dispensing movement of a plunger rod that contacts an audible clicker, as discussed above.

In step 812, the mobile device 840 pairs 844 with the autoinjector 850A. Optionally, an indicator light may indicate a status of the autoinjector 850A. In step 813, when turned on, the temperature sensor measures a temperature, and the wireless transfer protocol module can communicate 846 the temperature to the mobile device 840. The temperature sensor can provide any number and type of measurements, including a temperature reading or a rate of change of temperature (e.g., 0.01-0.2° C./minute, 0.01-0.1° C./minute, 0.02-0.2° C./minute, 0.02-0.1° C./minute, about 0.05° C./minute, etc.) determined by readings determined over a time interval (e.g., a time interval of one, two, three, four, or five minutes, in which measurements can be taken every 10, 20, 30, 45, 60, or more seconds). The temperature may be an ambient temperature determined within the autoinjector. While not a direct measurement, the ambient temperature can be used to infer the temperature of the medicament. The temperature may be of a medicament contained within the autoinjector. The user can wait until the medicament is at an appropriate temperature for injection. For example, in many cases, users keep autoinjectors in a refrigerator or other chilled location. After the autoinjector is removed from the refrigerator, the medicament warms up to an appropriate temperature for injection (e.g., 5-10° C., 5-20° C., 5-30° C., 10-20° C., 10-30° C., 20-30° C., less than 30° C., etc.). An indicator 871B on the autoinjector can indicate that the appropriate temperature has been reached.

When the medicament is at an appropriate temperature, the autoinjector 850A is ready for use. In step 814, the user can remove 860 a front cap 852 of the ready autoinjector 854. Removing the cap 852 of the autoinjector 854 reveals the needle guard 856 so that the needle can be inserted into the patient (see discussion below). Optionally, an indicator light 871C may indicate a status of the autoinjector 854.

In step 815, the user brings 861 the needle guard 856 into contact with his or her skin 801 to start the dose. The needle guard 856 is initially in an extended position 856A where the needle is covered. At step 816, the user inserts 862 the needle by pressing the autoinjector further into the skin. Inserting the needle into the patient's skin moves the needle guard 856 into a retracted position 856B. A time delay may be present between a time for inserting the needle into the skin and a time for beginning the dose of medicament. The internal components of the autoinjector inject the medicament into the user when the user inserts the needle into his or her skin (see discussion below). The user holds the autoinjector in place during the injection. In step 817, the dose ends after the medicament has been injected into the user and the autoinjector is removed 863 from the patient. An indicator 871F on the autoinjector can indicate that the dose is complete.

In step 818, after the dose ends, information about the dose can be displayed 848 on the mobile device. For example, information can include whether the dose was successfully injected, the time of the delivery, the amount of medicament delivered, a likely error in delivery (e.g., early lift off, shallow delivery, etc.). The information can be determined based on received signals from, e.g., the force sensor, the temperature sensor, the vibration sensor, the mobile device.

In step 819, the user can remove 864 the attachment module 870 from the housing of the autoinjector 854. An indicator 871G on the autoinjector can indicate that the attachment module 870 has been removed.

In step 820, the user can dispose 865 of the first autoinjector 850A. The user can dispose of the entire autoinjector or the housing of the autoinjector lacking the attachment module 870. Optionally, the user can remove the electronics from the autoinjector by removing the attachment module 870. In some non-limiting examples, the removed electronics can be disposed (e.g., to an appropriate waste stream). In other non-limiting examples, the removed electronics can be returned to the manufacturer for possible reassignment in a controlled manner.

In step 821, the user can attach 866 the attachment module 870 to the housing of a second autoinjector 850B. An indicator 871H on the autoinjector can indicate that the attachment module 870 has been attached. In step 822, the user can perform an injection with a second autoinjector 850B by performing 867 the portions of the method 800 described above. An indicator 871I on the autoinjector can indicate that the attachment module 870 is ready for use with the second autoinjector 850B.

In step 823, the user can dispose 870 of the attachment module 870. Optionally, the user can remove the electronics from the autoinjector by removing the attachment module 870. In some non-limiting examples, the removed electronics can be disposed (e.g., to an appropriate waste stream). In other non-limiting examples, the removed electronics can be returned to the manufacturer for possible reassignment in a controlled manner.

In step 824, the mobile device and/or another device, e.g., a device of a healthcare provider, can provide a full readout of information 880, 882 from the one or more injections performed by the user. For example, the full readout of information can include information about each of the injections performed by the user, including whether each dose was successfully injected, the time of the delivery, the amount of medicament delivered, etc.

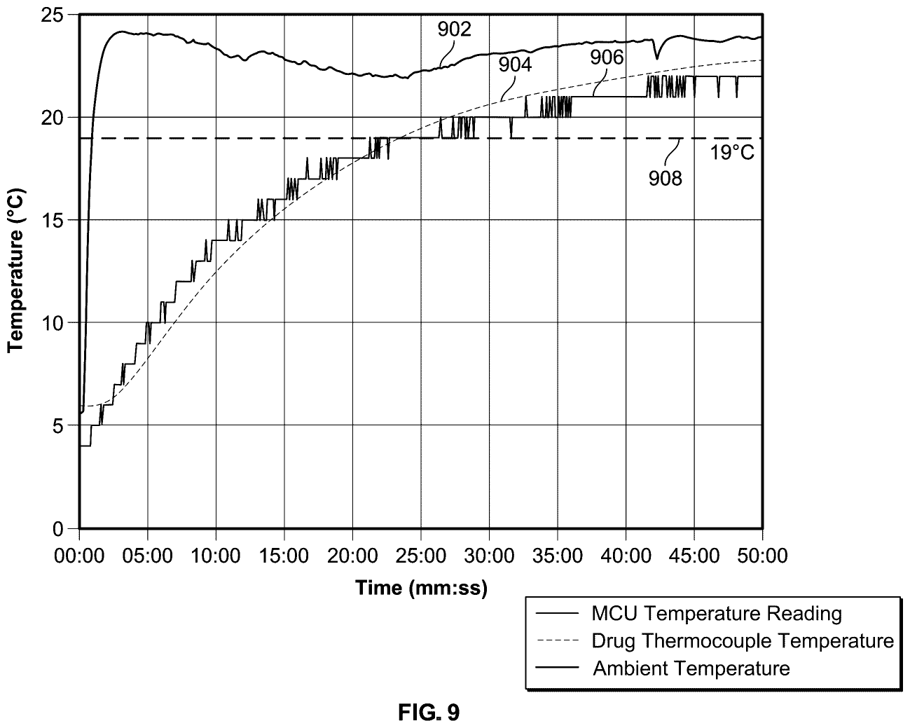

FIG. 9 is plot of temperature versus time. Three temperature traces are shown, ambient temperature 902, temperature of a drug 904 as measured by a thermocouple inserted into the drug, and a temperature reading 906 from a micro-control unit (MCU) with a thermistor. A threshold injection temperature 908 of 19° C. is also shown. When the drug is removed from a cooled location (e.g., a refrigerator), the ambient temperature quickly rises to normal room temperature. The MCU temperature reading 906 tracks the temperature of the drug 904 reasonably well indicating that the MCU temperature reading 906 is an adequate proxy for the temperature of the drug 904.

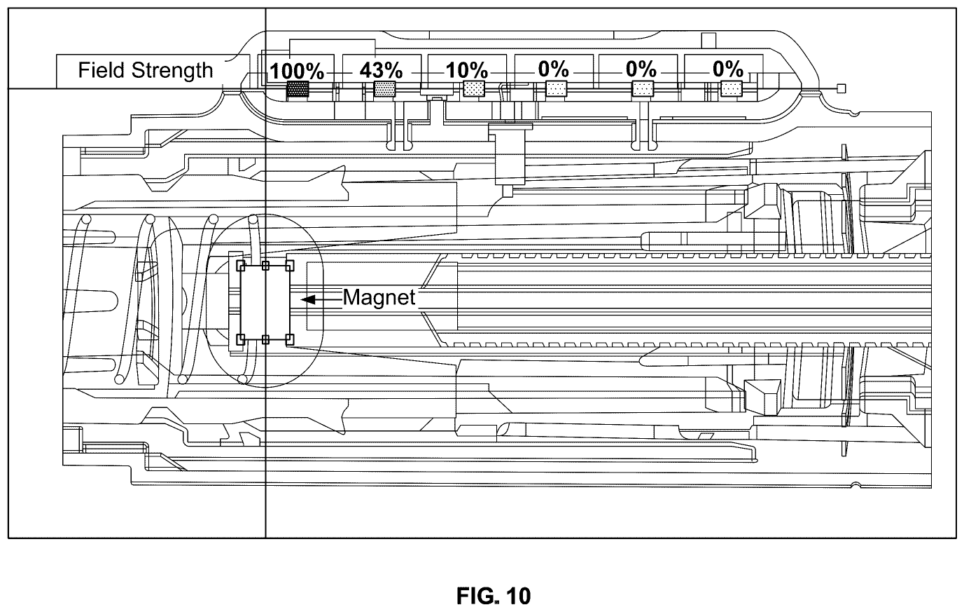

FIG. 10 is a plot of an example magnetic signal generated by an autoinjector indicating dose progress of the autoinjector during a dispensing movement of the autoinjector. Signals of higher magnetic field strength indicate closer proximity between the magnet on the plunger rod and the respective magnetic sensor (e.g., 100% or 43% field strength), whereas signals of lower magnetic field strength indicate increased distance between the magnet on the plunger rod and the respective magnetic sensor (e.g., 10% or 0% field strength). Depending on the types and arrangement of magnetic sensors and magnets, a skilled artisan can determine relevant thresholds for high and low magnetic field strength for determining dose progression. Optionally, if the autoinjector does not detect the end of dispensing movement signal, the autoinjector raises an error message that the dosing was incomplete. The error message can be generated and displayed by a connected device (e.g., a mobile device).

As used herein, the term “medicament” refers to a pharmaceutical formulation containing at least one active pharmaceutical ingredient (API) which is formulated for administration via injection (e.g., a liquid formulation). The API may be a compound which acts as an anti-viral or has anti-viral properties. The anti-viral API may be a compound that can treat or prevent viral disease such as HIV infection, hepatitis, or other viral disease. The anti-viral API of the medicament may treat or prevent HIV infection alone or in combination with other drugs.

The API may be an anti-HIV drug such as a capsid inhibitor (e.g., an antiretroviral drug that targets the capsid protein shell of viruses). Examples of suitable capsid inhibitors can be found, for example, in U.S. Pat. Nos. 9,951,043; 10,071,985; 11,944,611; 11,787,825; and 12,084,467; and U.S. Publication No.: 20230212148.

The capsid inhibitor may be lenacapavir sodium (SUNLENCA). As used herein, “lenacapavir” (or LEN) refers to N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide.

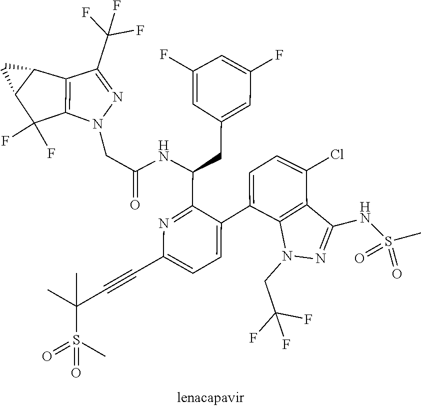 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US patent publications US20180051005 and US20190300505. Various forms and/or uses of lenacapavir are disclosed, for example, in US20190083478, US 20190084963, US20200038389A1, and US20210188815.

The capsid inhibitor may be lenacapavir, or a pharmaceutically acceptable salt thereof. The capsid inhibitor may be lenacapavir sodium. The capsid inhibitor may be lenacapavir (i.e., the free acid form of lenacapavir).

The present disclosure further includes medicaments (i.e., pharmaceutical compositions) comprising a capsid inhibitor provided herein, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the API, or a pharmaceutically acceptable salt thereof (e.g., for treatment or prevention of an HIV infection or reducing the risk of acquiring HIV).

Examples of suitable excipients are well known to the person skilled in the art of parenteral formulations and can be found, for example, in the Handbook of Pharmaceutical Excipients (eds. Rowe, Sheskey & Quinn), 6th edition 2009.

Examples of excipients in a parenteral formulation (for example, a subcutaneous or intramuscular formulation) include polyethylene glycol. In general, polyethylene glycol (PEG) is a polyether having a general formula H—(O—CH₂—CH₂)ₙ—OH. The PEG may be “capped” by an alkyl group. Optionally, the capped PEG may be of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. The average molecular weight of PEG within the pharmaceutical composition may be about 100 to about 800. The average molecular weight of PEG within the pharmaceutical composition may be about 200 to about 600. The average molecular weight of PEG within the pharmaceutical composition may be about 400. The average molecular weight of PEG within the pharmaceutical composition may be about 300. The average molecular weight of PEG within the pharmaceutical composition may be about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose, any bland fixed oil can be employed, including synthetic mono- or diglycerides. In addition, fatty acids such as oleic acid can likewise be used in the preparation of injectables.

Example formulations suitable for use with the systems and devices of the present application can be found, for example, in U.S. Pat. No. 11,807,625.

The pharmaceutical composition may comprise the API (e.g., lenacapavir or lenacapavir sodium) at a concentration of about 10 mg/mL to about 600 mg/mL. The pharmaceutical composition may comprise the API (e.g., lenacapavir or lenacapavir sodium) at a concentration of about 100 mg/mL to about 400 mg/mL. The pharmaceutical composition may comprise the API (e.g., lenacapavir or lenacapavir sodium) at a concentration of about 250 mg/mL to about 350 mg/mL. The pharmaceutical composition may comprise the API (e.g., lenacapavir or lenacapavir sodium) at a concentration of about 300 mg/mL to about 325 mg/mL. The pharmaceutical composition may comprise the API (e.g., lenacapavir or lenacapavir sodium) at a concentration of about 305 mg/mL to about 310 mg/mL. The pharmaceutical composition may comprise the API (e.g., lenacapavir or lenacapavir sodium) at a concentration of about 309 mg/mL.

The solution provided herein may comprise lenacapavir, or a pharmaceutically acceptable salt thereof, PEG 300, and water. The solution may comprise lenacapavir sodium, PEG 300, and water. The solution may comprise lenacapavir, PEG 300, and water.

The amount of water in the solution comprising lenacapavir sodium, PEG 300, and water may be about 10 w/w % to about 40 w/w %. The amount of water in the solution comprising lenacapavir sodium, PEG 300, and water may be about 15 w/w % to about 35 w/w %. The amount of water in the solution comprising lenacapavir sodium, PEG 300, and water may be about 20 w/w % to about 30 w/w %. The amount of water in the solution comprising lenacapavir sodium, PEG 300, and water may be about 21 w/w % to about 29 w/w %.

The amount of water in the solution comprising lenacapavir sodium, PEG 300, and water may be about 21.1 w/w % to about 27.5 w/w %. The amount of water in the solution comprising lenacapavir sodium, PEG 300, and water may be about 21.13 w/w % to about 27.47 w/w %. The amount of water in the solution comprising lenacapavir sodium, PEG 300, and water may be about 21.1 w/w %. The amount of water in the solution comprising lenacapavir sodium, PEG 300, and water may be about 21.13 w/w %. The amount of water in the solution comprising lenacapavir sodium, PEG 300, and water may be about 23 w/w %. The amount of water in the solution comprising lenacapavir sodium, PEG 300, and water may be about 23.4 w/w %. The amount of water in the solution comprising lenacapavir sodium, PEG 300, and water may be about 23.41 w/w %.

The amount of PEG 300 in the solution comprising lenacapavir sodium, PEG 300, and water may be about 35 w/w % to about 75 w/w %. The amount of PEG 300 in the solution comprising lenacapavir sodium, PEG 300, and water may be about 40 w/w % to about 55 w/w %. The amount of PEG 300 in the solution comprising lenacapavir sodium, PEG 300, and water may be about 43 w/w % to about 47 w/w %. The amount of PEG 300 in the solution comprising lenacapavir sodium, PEG 300, and water may be about 45 w/w %. The amount of PEG 300 in the solution comprising lenacapavir sodium, PEG 300, and water may be about 45.25 w/w %. The amount of PEG 300 in the solution comprising lenacapavir sodium, PEG 300, and water may be about 48 w/w % to about 52 w/w %. The amount of PEG 300 in the solution comprising lenacapavir sodium, PEG 300, and water may be about 50 w/w %. The amount of PEG 300 in the solution comprising lenacapavir sodium, PEG 300, and water may be about 50.1 w/w %. The amount of PEG 300 in the solution comprising lenacapavir sodium, PEG 300, and water may be about 50.13 w/w %.

The amount of lenacapavir sodium in the solution comprising PEG 300 and water may be about 5 w/w % to about 35 w/w %. The amount of lenacapavir sodium in the solution comprising PEG 300 and water may be about 20 w/w % to about 35 w/w %.

The amount of lenacapavir sodium in the solution comprising PEG 300 and water may be about 20 w/w % to about 30 w/w %. The amount of lenacapavir sodium in the solution comprising PEG 300 and water may be about 24 w/w % to about 28 w/w %. The amount of lenacapavir sodium in the solution comprising PEG 300 and water may be about 26.5 w/w %. The amount of lenacapavir sodium in the solution comprising PEG 300 and water may be about 26.46 w/w %.

The amount of lenacapavir sodium in the solution comprising PEG 300 and water may be about 30 w/w % to about 35 w/w %. The amount of lenacapavir sodium in the solution comprising PEG 300 and water may be about 32 w/w % to about 34 w/w %. The amount of lenacapavir sodium in the solution comprising PEG 300 and water may be about 33.6 w/w %. The amount of lenacapavir sodium in the solution comprising PEG 300 and water may be about 33.61 w/w %.

The solution may comprise about 10 w/w % to about 40 w/w % water, about 35 w/w % to about 75 w/w % PEG 300, and about 5 w/w % to about 45 w/w % of lenacapavir sodium. The solution may comprise about 10 w/w % to about 30 w/w % water, about 35 w/w % to about 65 w/w % PEG 300, and about 5 w/w % to about 45 w/w % of lenacapavir sodium.

The solution may comprise about 21.13 w/w % to about 27.47 w/w % water, about 45.25 w/w % to about 58.84 w/w % PEG 300, and about 13.69 w/w % to about 33.61 w/w % of lenacapavir sodium. The solution may comprise about 21.1 w/w % water, about 45.3 w/w % PEG 300, and about 33.6 w/w % of lenacapavir sodium. The solution may comprise about 21.13 w/w % water, about 45.25 w/w % PEG 300, and about 33.61 w/w % of lenacapavir sodium. The solution may comprise about 23.4 w/w % water, about 50.1 w/w % PEG 300, and about 26.5 w/w % of lenacapavir sodium. The solution may comprise about 23.41 w/w % water, about 50.13 w/w % PEG 300, and about 26.46 w/w % of lenacapavir sodium.

The solution provided herein may further comprise ethanol. The solution may comprise about 10 w/w % to about 40 w/w % water, about 20 w/w % to about 75 w/w % PEG 300, about 10 w/w % to about 70 w/w % of lenacapavir sodium, and about 1 w/w % to about 10 w/w % of ethanol. The solution may comprise about 10 w/w % to about 20 w/w % water, about 30 w/w % to about 40 w/w % PEG 300, about 37 w/w % to about 45 w/w % of lenacapavir sodium, and about 3 w/w % to about 8 w/w % of ethanol.

The solution may comprise about 16.93 w/w % water, about 36.22 w/w % PEG 300, about 41.85 w/w % of lenacapavir sodium, and about 5.00 w/w % ethanol. The solution may comprise about 16.9 w/w % water, about 36.2 w/w % PEG 300, about 41.9 w/w % of lenacapavir sodium, and about 5.0 w/w % ethanol.

The medicament which is administered by the injection device of the invention may be a long-acting injectable formulation which can be administered to a patient, for example, twice per month, once per month, once per quarter (e.g., every 3 months), twice per year (e.g., every 6 months), once per year, or less frequently.

Additionally, the medicament can include a formulation which has relatively high viscosity. The medicament may have a viscosity of 100-1000 cP. Preferably, the medicament may have a viscosity of 100-600 cP. For example, the medicament may have a viscosity of 250-500 cP, 150-350 cP or 450-600 cP. The medicament may be a solution comprising lenacapavir sodium the medicament may have a viscosity of 250-500 cP, 150-350 cP or 450-600 cP.

The sensing assembly (e.g., the main PCB of autoinjectors 100, 200, or 300, which may be provided within an attachable module) is intended to include various forms of digital computers, such as printed circuit boards (PCB), processors, digital circuitry, or otherwise parts of a system for determining dosing progression of an autoinjector. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The sensing assembly can include a processor, a memory, a storage device, and an input/output device (for example, sensors). Each of the components is interconnected using a system bus. The sensing assembly is capable of processing instructions for execution within the sensing assembly. The sensing assembly may be designed using any of a number of architectures. For example, the sensing assembly can include a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on an input/output device.

The memory stores information within the sensing assembly. In one implementation, the memory is a computer-readable medium. In one implementation, the memory is a volatile memory unit. In another implementation, the memory is a non-volatile memory unit.

The storage device is capable of providing mass storage for the sensing assembly. In one implementation, the storage device is a computer-readable medium. In various different implementations, the storage device may be a floppy disk device, a hard disk device, an optical disk device, or a tape device.

The features described can be implemented in digital electronic circuitry, or in computer hardware, firmware, software, or in combinations of them. The features can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

A number of embodiments have been described. Nevertheless, it will be understood that various modifications may be made without departing from the spirit and scope of the disclosure. Accordingly, other embodiments are within the scope of the following claims.

### Embodiments

The following list provides embodiments of the invention and forms part of the description. These embodiments can be combined in any compatible combination beyond those expressly stated. The embodiments can also be combined with any compatible features described herein:
1. An autoinjector comprising:
   a housing;
   a needle arranged at a distal end of the housing;
   a container disposed within the housing and configured to contain medicament;
   a plunger slidably disposed within the container;
   a plunger rod configured to push the plunger through the container to dispense the medicament through the needle when the container contains medicament, wherein the plunger rod comprises a magnetic region, and wherein a dispensing movement of the plunger rod generates a first magnetic signal; and
   a first sensor configured to detect said first magnetic signal as the plunger rod moves.
2. The autoinjector of embodiment 1, wherein the first magnetic signal is indicative of dose progression.
3. The autoinjector of any one of embodiment 1 or embodiment 2, wherein the first magnetic signal is configured to indicate completion of the dispensing movement of the plunger rod.
4. The autoinjector of any of embodiments 1-3, wherein the container contains the medicament.
5. The autoinjector of any preceding embodiment, wherein the medicament comprises lenacapavir or a pharmaceutically accepted salt thereof.
6. The autoinjector of embodiment 5, wherein the medicament comprises lenacapavir sodium.
7. The autoinjector of embodiment 6, wherein the medicament is a solution comprising: lenacapavir sodium, PEG 300 and water.
8. The autoinjector of embodiment 7, wherein the solution comprises about 21.13 w/w % water, about 45.25 w/w % PEG 300, and about 33.61 w/w % of lenacapavir sodium.
9. The autoinjector of embodiment 7, wherein the solution comprises about 23.41 w/w % water, about 50.13 w/w % PEG 300, and about 26.46 w/w % of lenacapavir sodium.
10. The autoinjector of embodiment 7, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.
11. The autoinjector of any preceding embodiment, wherein the autoinjector further comprises a gas canister assembly configured to release pressurized gas which, when released, provides a force acting on the plunger rod to push the plunger through the container.
12. The autoinjector of any of embodiments 1-11, wherein the magnetic region is disposed on a distal end of the plunger rod.
13. The autoinjector of any of embodiments 1-12, wherein the magnetic region comprises a magnet.
14. The autoinjector of any one of embodiments 0-13, wherein the plunger rod is configured to generate a plurality of magnetic signals during the dispensing movement of the plunger rod.
15. The autoinjector of embodiment 14, wherein the first sensor comprises an array of sensor units.
16. The autoinjector of embodiment 15, wherein each of the sensor units comprises, independently, a Hall effect sensor.
17. The autoinjector of embodiment 15 or embodiment 16, wherein the array comprises a first sensor unit disposed in proximity to a proximal end of the container and a last sensor unit disposed in proximity to a distal end of the container.
18. The autoinjector of embodiment 17, wherein the first sensor unit is configured to indicate dose start of the medicament, and/or wherein the last sensor unit is configured to indicate dose end of the medicament.
19. The autoinjector of embodiment 1, wherein the magnetic region is configured to generate a corresponding magnetic signal during the dispensing movement of the plunger rod.
20. The autoinjector of any one of embodiments 0-20, further comprising:
   an audible clicker configured to produce one or more audible clicks during the dispensing movement of the plunger rod.
21. The autoinjector of embodiment 20, wherein the audible clicker comprises a ring surrounding the plunger rod, and wherein the ring comprises a deflectable protrusion configured to contact the plunger rod and produce the one or more audible clicks.
22. The autoinjector of embodiment 21, wherein the plunger rod comprises a ridged surface configured to contact the deflectable protrusion of the audible clicker.
23. The autoinjector of embodiment 22, wherein each ridge of the ridged surface is configured to cause the deflectable protrusion to deflect during the dispensing movement of the plunger rod.
24. The autoinjector of any one of embodiments 1-23, wherein the first sensor is disposed in an attachable module configured to be reversibly attached to the housing.
25. The autoinjector of embodiment 24, wherein the attachable module comprises an assembly switch configured to detect attachment of the attachable module to the housing.
26. The autoinjector of embodiment 25, wherein the assembly switch is configured to detect attachment of the attachable module to the housing and is further configured to provide power to one or more electronic components of the autoinjector.
27. The autoinjector of any of embodiments 1-24, further comprising an integrated sensor disposed between a proximal end and a distal end of the housing, wherein the integrated sensor comprises a second sensor configured to detect a first electrical signal generated by applying an insertion force to insert the needle into a user and a third sensor is configured to detect an ambient temperature in proximity to the container.
28. The autoinjector of embodiment 27, further comprising a needle guard contact configured to generate the first electrical signal upon applying the insertion force.
29. The autoinjector of any of embodiments 1-26, further comprising a second sensor disposed between a proximal end and a distal end of the housing, wherein the second sensor is configured to detect a first electrical signal generated by applying an insertion force to insert the needle into a user.
30. The autoinjector of embodiment 29, further comprising a needle guard contact configured to generate the first electrical signal upon applying the insertion force.
31. The autoinjector of embodiment 29 or embodiment 30, wherein the second sensor comprises a force sensor.
32. The autoinjector of any of embodiments 1-26 or 29-31, further comprising a third sensor disposed between a proximal end and a distal end of the housing, wherein the third sensor is configured to detect an ambient temperature in proximity to the container.
33. The autoinjector of embodiment 32, wherein the third sensor comprises a thermistor.
34. An autoinjector comprising:
   a housing;
   a needle arranged at a distal end of the housing;
   a container disposed within the housing and configured to contain medicament;
   a plunger slidably disposed within the container;
   a plunger rod configured to push the plunger through the container to dispense the medicament through the needle when the container contains medicament, wherein the plunger rod comprises a magnetic region;
   a mechanism configured so that a dispensing movement of the plunger rod causes the mechanism to generate a first magnetic signal; and
   a first sensor configured to detect said first magnetic signal generated by the mechanism.
35. An autoinjector comprising:
   a housing comprising a recessed portion;
   a needle arranged at a distal end of the housing;
   a needle guard configured to expose the needle when an insertion force is applied to insert the needle into a user;
   a container disposed within the housing and configured to contain medicament;
   a plunger slidably disposed within the container;
   a plunger rod configured to push the plunger through the container to dispense the medicament through the needle when the container contains medicament and when the needle is exposed, wherein the plunger rod comprises a magnetic region, and wherein a dispensing movement of the plunger rod generates a first magnetic signal; and
   an attachable module configured to be reversibly attached to the recessed portion of the housing, wherein the attachable module comprises a first sensor configured to detect said first magnetic signal as the plunger rod moves.
36. A system comprising:
   an autoinjector of any one of embodiments 1-35; and
   a processor configured to process one or more magnetic signals to track the dispensing movement of the plunger rod.
37. A method of detecting a dispensing movement of a plunger rod within an autoinjector, the method comprising:
   detecting one or more magnetic signals of a magnetic region as the plunger rod moves during the dispensing movement, wherein the plunger rod comprises the magnetic region disposed on or within the plunger rod, and
   wherein the dispensing movement of the plunger rod generates a first magnetic signal.
38. A method of detecting a dispensing movement of a plunger rod within an autoinjector, the method comprising:
   detecting a magnetic signal generated due to movement of the plunger rod during the dispensing movement.
39. The method of embodiment 37 or 38, wherein the plunger rod is configured to generate a plurality of magnetic signals during the dispensing movement of the plunger rod.
40. The method of any one of embodiments 37-39, wherein the autoinjector further comprises an audible clicker configured to produce one or more audible clicks during the dispensing movement of the plunger rod.
41. The method of embodiment 40, wherein the audible clicker comprises a ring surrounding the plunger rod, and wherein the ring comprises a deflectable protrusion configured to contact the plunger rod and produce the one or more audible clicks.
42. The method of embodiment 41, wherein the plunger rod comprises a ridged surface configured to contact the deflectable protrusion of the audible clicker.
43. The method of embodiment 42, wherein each ridge of the ridged surface is configured to cause the deflectable protrusion to deflect during the dispensing movement of the plunger rod.
44. The method of any of embodiments 37-43, wherein a first sensor is disposed between a proximal end and a distal end of the housing.
45. The method of embodiment 44, wherein the first sensor comprises a magnetic sensor or an array of magnetic sensors.
46. The method of embodiment 44 or embodiment 45, wherein the first sensor is disposed on a surface of a printed circuit board.
47. The method of any of embodiments 37-46, wherein an integrated sensor is disposed between a proximal end and a distal end of the housing, and wherein the integrated sensor comprises a second sensor configured to detect a first electrical signal generated by applying an insertion force to insert the needle into a user and a third sensor configured to detect an ambient temperature in proximity to the container.
48. The method of any of embodiments 37-46, wherein a second sensor is disposed between a proximal end and a distal end of the housing, and wherein the second sensor is configured to detect a first electrical signal generated by applying an insertion force to insert the needle into a user.
49. The method of any of embodiments 37-46 or 48, wherein a third sensor is disposed between a proximal end and a distal end of the housing, and wherein the third sensor is configured to detect an ambient temperature in proximity to the container.
50. The method of any one of embodiments 37-49, further comprising sending, via a wireless transfer protocol module, signals from a first sensor, if present, an integrated sensor, if present, and/or a second sensor, if present, to a mobile device.
51. The method of embodiment 50, further comprising displaying information about the dispensing movement on the mobile device.
52. The method of any one of embodiments 37-51, further comprising measuring a temperature of medicament within the autoinjector or an ambient temperature within the autoinjector using a temperature sensor.
53. The method of embodiment 52, further comprising sending, via a wireless transfer protocol module, signals from the temperature sensor to a mobile device.
54. The method of embodiment 52 or embodiment 53, further comprising displaying the temperature of the medicament or the ambient temperature on the mobile device; indicating that the temperature of the medicament or the ambient temperature is above a threshold temperature for use of the autoinjector; or both.
55. A composition comprising lenacapavir or a pharmaceutically accepted salt thereof for use in the prevention or treatment of HIV, wherein the composition is administered via the autoinjector of any of embodiments 1 to 36.
56. The composition of embodiment 55, wherein the composition is subcutaneously administered via the autoinjector, optionally wherein the medicament is a solution comprising either:
   (a) about 21.13 w/w % water, about 45.25 w/w % PEG 300, and about 33.61 w/w % of lenacapavir sodium; or
   (b) about 23.41 w/w % water, about 50.13 w/w % PEG 300, and about 26.46 w/w % of lenacapavir sodium.
57. The composition of embodiment 55, wherein the composition is intramuscularly administered via the autoinjector, optionally wherein the composition is a solution comprising about 16.9 w/w % water, about 36.2 w/w % PEG 300, about 41.9 w/w % of lenacapavir sodium, and about 5.0 w/w % ethanol.
58. Use of lenacapavir or a pharmaceutically accepted salt thereof for the manufacture of a medicament for the prevention or treatment of HIV, wherein the prevention or treatment comprises administering the medicament via the autoinjector of any of embodiments 1 to 36.
59. The use of embodiment 58, wherein the medicament is subcutaneously administered via the autoinjector, optionally wherein the medicament is a solution comprising either:
   (a) about 21.13 w/w % water, about 45.25 w/w % PEG 300, and about 33.61 w/w % of lenacapavir sodium; or
   (b) about 23.41 w/w % water, about 50.13 w/w % PEG 300, and about 26.46 w/w % of lenacapavir sodium.
60. The use of embodiment 58, wherein the medicament is intramuscularly administered via the autoinjector, optionally wherein the medicament is a solution comprising about 16.9 w/w % water, about 36.2 w/w % PEG 300, about 41.9 w/w % of lenacapavir sodium, and about 5.0 w/w % ethanol.

1. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises a magnetic region, and wherein a dispensing movement of the plunger rod generates a first magnetic signal; and
a first sensor configured to detect said first magnetic signal as the plunger rod moves.

2. The autoinjector of claim 1, wherein the first magnetic signal is indicative of dose progression.

3. The autoinjector of any one of claim 1, wherein the first magnetic signal is configured to indicate completion of the dispensing movement of the plunger rod.

4. (canceled)

5. The autoinjector of claim 1, wherein the medicament comprises lenacapavir or a pharmaceutically accepted salt thereof.

6. The autoinjector of claim 1, wherein the autoinjector further comprises a gas canister assembly configured to release pressurized gas which, when released, provides a force acting on the plunger rod to push the plunger through the container.

7. The autoinjector of claim 1, wherein the magnetic region is disposed on a distal end of the plunger rod.

8-9. (canceled)

10. The autoinjector of claim 1, wherein the first sensor comprises an array of sensor units and the plunger rod is configured to generate a plurality of magnetic signals during the dispensing movement of the plunger rod.

11. The autoinjector of claim 10, wherein each of the sensor units comprises, independently, a Hall effect sensor.

12. The autoinjector of claim 10, wherein the array comprises a first sensor unit disposed in proximity to a proximal end of the container and a last sensor unit disposed in proximity to a distal end of the container.

13. The autoinjector of claim 12, wherein the first sensor unit is configured to indicate dose start of the medicament, and/or wherein the last sensor unit is configured to indicate dose end of the medicament.

14. (canceled)

15. The autoinjector of claim 1, further comprising:
an audible clicker comprising a ring surrounding the plunger rod and the ring comprises a deflectable protrusion configured to contact the plunger rod to produce one or more audible clicks during the dispensing movement of the plunger rod.

16. (canceled)

17. The autoinjector of claim 15, wherein the plunger rod comprises a ridged surface configured to cause the deflectable protrusion of the audible clicker to deflect during the dispensing movement of the plunger rod.

18. (canceled)

19. The autoinjector of claim 1, wherein the first sensor is disposed in an attachable module configured to be reversibly attached to the housing.

20. The autoinjector of claim 19, wherein the attachable module comprises an assembly switch configured to detect attachment of the attachable module to the housing.

21. The autoinjector of claim 20, wherein the assembly switch is configured to detect attachment of the attachable module to the housing and is further configured to provide power to one or more electronic components of the autoinjector.

22-23. (canceled)

24. The autoinjector of claim 19, further comprising a second sensor disposed between a proximal end and a distal end of the housing, wherein the second sensor is configured to detect a first electrical signal generated by applying an insertion force to insert the needle into a user.

25. The autoinjector of claim 24, further comprising a needle guard contact configured to generate the first electrical signal upon applying the insertion force.

26. (canceled)

27. The autoinjector of claim 24, further comprising a third sensor disposed between a proximal end and a distal end of the housing, wherein the third sensor is configured to detect an ambient temperature in proximity to the container.

28-29. (canceled)

30. An autoinjector comprising:
a housing comprising a recessed portion;
a needle arranged at a distal end of the housing;
a needle guard configured to expose the needle when an insertion force is applied to insert the needle into a user;
a container disposed within the housing and configured to contain medicament;
a plunger slidably disposed within the container;
a plunger rod configured to push the plunger through the container to dispense the medicament through the needle when the container contains medicament and when the needle is exposed, wherein the plunger rod comprises a magnetic region, and wherein a dispensing movement of the plunger rod generates a first magnetic signal; and
an attachable module configured to be reversibly attached to the recessed portion of the housing, wherein the attachable module comprises a first sensor configured to detect said first magnetic signal as the plunger rod moves.

31. (canceled)

32. A method of detecting a dispensing movement of a plunger rod within an autoinjector, the method comprising:
detecting one or more magnetic signals of a magnetic region as the plunger rod moves during the dispensing movement, wherein the plunger rod comprises the magnetic region disposed on or within the plunger rod, and
wherein the dispensing movement of the plunger rod generates a first magnetic signal.

33. (canceled)

34. The method of claim 32, wherein the plunger rod is configured to generate a plurality of magnetic signals during the dispensing movement of the plunger rod.

35. The method of claim 32, wherein the autoinjector further comprises an audible clicker, and the method further comprises producing one or more audible clicks during the dispensing movement of the plunger rod.

36-38. (canceled)

39. The method of claim 32, wherein a first sensor is disposed between a proximal end and a distal end of a housing, and wherein the first sensor comprises a magnetic sensor or an array of magnetic sensors.

40-42. (canceled)

43. The method of claim 39, wherein a second sensor is disposed between a proximal end and a distal end of the housing, and wherein the second sensor is configured to detect a first electrical signal generated by applying an insertion force to insert a needle into a user.

44. The method of claim 39, wherein a third sensor is disposed between a proximal end and a distal end of the housing, and wherein the third sensor is configured to detect an ambient temperature in proximity to a container.

45. The method of claim 44, further comprising sending, via a wireless transfer protocol module, signals from the first sensor the second sensor, and/or the third sensor to a mobile device.

46. The method of claim 45, further comprising displaying information about the dispensing movement on the mobile device.

47. The method of claim 32, further comprising measuring a temperature of medicament within the autoinjector or an ambient temperature within the autoinjector using a temperature sensor.

48. The method of claim 47, further comprising sending, via a wireless transfer protocol module, signals from the temperature sensor to a mobile device.

49. The method of claim 47, further comprising displaying the temperature of the medicament or the ambient temperature on a mobile device; indicating that the temperature of the medicament or the ambient temperature is above a threshold temperature for use of the autoinjector; or both.

50. A composition comprising lenacapavir or a pharmaceutically accepted salt thereof for use in the prevention or treatment of HIV, wherein the composition is administered via the autoinjector of claim 1.

51. Use of lenacapavir or a pharmaceutically accepted salt thereof for the manufacture of a medicament for the prevention or treatment of HIV, wherein the prevention or treatment comprises administering the medicament via the autoinjector of claim 1.
